SEQUENCE LISTING (30 sequences)

SEQ ID NO: 1 - protein, 268 aa, Streptomyces rimosus
MRLSRRAATASALLLTPALALFGASAAVSAPRIQATDYVALGDSYSSGVGAGSYDSSSGSCKRSTKSYPALWAASHTGTRFNFTACSGARTGDVLAKQLTPVNSGTDLVSITIGGNDAGFADTMTTCNLQGESACLARIAKARAYIQQTLPAQLDQVYDAIDSRAPAAQVVVLGYPRFYKLGGSCAVGLSEKSRAAINAAADDINAVTAKRAADHGFAFGDVNTTFAGHELCSGAPWLHSVTLPVENSYHPTANGQSKGYLPVLNSAT

SEQ ID NO: 2 - protein, 240 aa, Streptomyces rimosus
SAPRIQATDYVALGDSYSSGVGAGSYDSSSGSCKRSTKSYPALWAASHTGTRFNFTACSGARTGDVLAKQLTPVNSGTDLVSITIGGNDAGFADTMTTCNLQGESACLARIAKARAYIQQTLPAQLDQVYDAIDSRAPAAQVVVLGYPRFYKLGGSCAVGLSEKSRAAINAAADDINAVTAKRAADHGFAFGDVNTTFAGHELCSGAPWLHSVTLPVENSYHPTANGQSKGYLPVLNSAT

SEQ ID NO: 3 - protein, 286 aa, Artificial Sequence
  Chemically synthesized amino acid sequence of CelA signal sequence-SriII fusion protein
MGFGSAPIALCPLRTRRNALKRLLALLATGVSIVGLTALAGPPAQASAPRIQATDYVALGDSYSSGVGAGSYDSSSGSCKRSTKSYPALWAASHTGTRFNFTACSGARTGDVLAKQLTPVNSGTDLVSITIGGNDAGFADTMTTCNLQGESACLARIAKARAYIQQTLPAQLDQVYDAIDSRAPAAQVVVLGYPRFYKLGGSCAVGLSEKSRAAINAAADDINAVTAKRAADHGFAFGDVNTTFAGHELCSGAPWLHSVTLPVENSYHPTANGQSKGYLPVLNSAT

SEQ ID NO: 4 - DNA, 807 bp, Streptomyces rimosus
atgcgcctgtcccgccgcgccgccaccgcctccgccctgctgctgaccccggccctggccctgttcggcgcctccgccgccgtcagcgccccgcgcatccaggccaccgactacgtggccctcggcgactcctactcctcgggcgtgggcgccggctcctacgacagctccagcggctcctgcaagcgctccaccaagtcctacccggccctgtgggccgcctcccacaccggcacccgcttcaacttcaccgcctgctccggcgcccgcaccggcgacgtcctggccaagcagctgaccccggtcaactccggcaccgacctggtctccatcaccatcggcggcaacgacgccggcttcgccgacaccatgaccacctgcaacctgcagggcgagtccgcctgcctggcccgcatcgccaaggcccgcgcctacatccagcagaccctgccggcccagctggaccaggtctacgacgccatcgactcccgcgccccggccgcgcaggtcgtcgtgctgggctacccgcgcttctacaagctgggcggctcctgcgccgtcggcctgtccgagaagtcccgcgccgccatcaacgccgccgccgacgacatcaacgccgtcaccgccaagcgcgccgccgaccacggcttcgccttcggcgacgtcaacaccaccttcgccggccacgagctgtgctcgggcgccccgtggctgcactccgtcaccctcccggtcgagaacagctaccacccgaccgccaacggccagtccaagggctacctgccggtcctgaactccgccacctga

SEQ ID NO: 5 - protein, 295 aa, Streptomyces coelicolor
MPKPALRRVMTATVAAVGTLALGLTDATAHAAPAQATPTLDYVALGDSYSAGSGVLPVDPANLLCLRSTANYPHVIADTTGARLTDVTCGAAQTADFTRAQYPGVAPQLDALGTGTDLVTLTIGGNDNSTFINAITACGTAGVLSGGKGSPCKDRHGTSFDDEIEANTYPALKEALLGVRARAPHARVAALGYPWITPATADPSCFLKLPLAAGDVPYLRAIQAHLNDAVRRAAEETGATYVDFSGVSDGHDACEAPGTRWIEPLLFGHSLVPVHPNALGERRMAEHTMDVLGLD

SEQ ID NO: 6 - protein, 264 aa, Streptomyces coelicolor
APAQATPTLDYVALGDSYSAGSGVLPVDPANLLCLRSTANYPHVIADTTGARLTDVTCGAAQTADFTRAQYPGVAPQLDALGTGTDLVTLTIGGNDNSTFINAITACGTAGVLSGGKGSPCKDRHGTSFDDEIEANTYPALKEALLGVRARAPHARVAALGYPWITPATADPSCFLKLPLAAGDVPYLRAIQAHLNDAVRRAAEETGATYVDFSGVSDGHDACEAPGTRWIEPLLFGHSLVPVHPNALGERRMAEHTMDVLGLD

SEQ ID NO: 7 - protein, 310 aa, Artificial Sequence
  Chemically synthesized amino acid sequence of CelA signal sequence-ScoIIA fusion protein
MGFGSAPIALCPLRTRRNALKRLLALLATGVSIVGLTALAGPPAQAAPAQATPTLDYVALGDSYSAGSGVLPVDPANLLCLRSTANYPHVIADTTGARLTDVTCGAAQTADFTRAQYPGVAPQLDALGTGTDLVTLTIGGNDNSTFINAITACGTAGVLSGGKGSPCKDRHGTSFDDEIEANTYPALKEALLGVRARAPHARVAALGYPWITPATADPSCFLKLPLAAGDVPYLRAIQAHLNDAVRRAAEETGATYVDFSGVSDGHDACEAPGTRWIEPLLFGHSLVPVHPNALGERRMAEHTMDVLGLD

SEQ ID NO: 8 - DNA, 933 bp, Streptomyces coelicolor
atgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggccgcgcccgcccaggccactccgaccctggactacgtcgccctcggcgacagctacagcgccggctccggcgtcctgcccgtcgaccccgccaacctgctctgtctgcgctcgacggccaactacccccacgtcatcgcggacacgacgggcgcccgcctcacggacgtcacctgcggcgccgcgcagaccgccgacttcacgcgggcccagtacccgggcgtcgcaccccagttggacgcgctcggcaccggcacggacctggtcacgctcaccatcggcggcaacgacaacagcaccttcatcaacgccatcacggcctgcggcacggcgggtgtcctcagcggcggcaagggcagcccctgcaaggacaggcacggcacctccttcgacgacgagatcgaggccaacacgtaccccgcgctcaaggaggcgctgctcggcgtccgcgccagggctccccacgccagggtggcggctctcggctacccgtggatcaccccggccaccgccgacccgtcctgcttcctgaagctccccctcgccgccggtgacgtgccctacctgcgggccatccaggcacacctcaacgacgcggtccggcgggccgccgaggagaccggagccacctacgtggacttctccggggtgtccgacggccacgacgcctgcgaggcccccggcacccgctggatcgaaccgctgctcttcgggcacagcctcgttcccgtccaccccaacgccctgggcgagcggcgcatggccgagcacacgatggacgtcctcggcctggactga

SEQ ID NO: 9 - protein, 268 aa, Streptomyces coelicolor
MRRFRLVGFLSSLVLAAGAALTGAATAQAAQPAAADGYVALGDSYSSGVGAGSYISSSGDCKRSTKAHPYLWAAAHSPSTFDFTACSGARTGDVLSGQLGPLSSGTGLVSISIGGNDAGFADTMTTCVLQSESSCLSRIATAEAYVDSTLPGKLDGVYSAISDKAPNAHVVVIGYPRFYKLGTTCIGLSETKRTAINKASDHLNTVLAQRAAAHGFTFGDVRTTFTGHELCSGSPWLHSVNWLNIGESYHPTAAGQSGGYLPVLNGAA

SEQ ID NO: 10 - protein, 239 aa, Streptomyces coelicolor
AQPAAADGYVALGDSYSSGVGAGSYISSSGDCKRSTKAHPYLWAAAHSPSTFDFTACSGARTGDVLSGQLGPLSSGTGLVSISIGGNDAGFADTMTTCVLQSESSCLSRIATAEAYVDSTLPGKLDGVYSAISDKAPNAHVVVIGYPRFYKLGTTCIGLSETKRTAINKASDHLNTVLAQRAAAHGFTFGDVRTTFTGHELCSGSPWLHSVNWLNIGESYHPTAAGQSGGYLPVLNGAA

SEQ ID NO: 11 - protein, 285 aa, Artificial Sequence
  Chemically synthesized amino acid sequence of CelA signal sequence-ScoIIB fusion protein
MGFGSAPIALCPLRTRRNALKRLLALLATGVSIVGLTALAGPPAQAAQPAAADGYVALGDSYSSGVGAGSYISSSGDCKRSTKAHPYLWAAAHSPSTFDFTACSGARTGDVLSGQLGPLSSGTGLVSISIGGNDAGFADTMTTCVLQSESSCLSRIATAEAYVDSTLPGKLDGVYSAISDKAPNAHVVVIGYPRFYKLGTTCIGLSETKRTAINKASDHLNTVLAQRAAAHGFTFGDVRTTFTGHELCSGSPWLHSVNWLNIGESYHPTAAGQSGGYLPVLNGAA

SEQ ID NO: 12 - DNA, 858 bp, Streptomyces coelicolor
atgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggccgcccaacccgccgccgccgacggctatgtggccctcggcgactcctactcctccggggtcggagcgggcagctacatcagctcgagcggcgactgcaagcgcagcacgaaggcccatccctacctgtgggcggccgcccactcgccctccacgttcgacttcaccgcctgttccggcgcccgtacgggtgatgttctctccggacagctcggcccgctcagctccggcaccggcctcgtctcgatcagcatcggcggcaacgacgccggtttcgccgacaccatgacgacctgtgtgctccagtccgagagctcctgcctgtcgcggatcgccaccgccgaggcgtacgtcgactcgacgctgcccggcaagctcgacggcgtctactcggcaatcagcgacaaggcgccgaacgcccacgtcgtcgtcatcggctacccgcgcttctacaagctcggcaccacctgcatcggcctgtccgagaccaagcggacggcgatcaacaaggcctccgaccacctcaacaccgtcctcgcccagcgcgccgccgcccacggcttcaccttcggcgacgtacgcaccaccttcaccggccacgagctgtgctccggcagcccctggctgcacagcgtcaactggctgaacatcggcgagtcgtaccaccccaccgcggccggccagtccggtggctacctgccggtcctcaacggcgccgcctga

SEQ ID NO: 13 - protein, 300 aa, Corynebacterium efficiens
MRTTVIAASALLLLAGCADGAREETAGAPPGESSGGIREEGAEASTSITDVYIALGDSYAAMGGRDQPLRGEPFCLRSSGNYPELLHAEVTDLTCQGAVTGDLLEPRTLGERTLPAQVDALTEDTTLVTLSIGGNDLGFGEVAGCIRERIAGENADDCVDLLGETIGEQLDQLPPQLDRVHEAIRDRAGDAQVVVTGYLPLVSAGDCPELGDVSEADRRWAVELTGQINETVREAAERHDALFVLPDDADEHTSCAPPQQRWADIQGQQTDAYPLHPTSAGHEAMAAAVRDALGLEPVQP

SEQ ID NO: 14 - protein, 279 aa, Corynebacterium efficiens
REETAGAPPGESSGGIREEGAEASTSITDVYIALGDSYAAMGGRDQPLRGEPFCLRSSGNYPELLHAEVTDLTCQGAVTGDLLEPRTLGERTLPAQVDALTEDTTLVTLSIGGNDLGFGEVAGCIRERIAGENADDCVDLLGETIGEQLDQLPPQLDRVHEAIRDRAGDAQVVVTGYLPLVSAGDCPELGDVSEADRRWAVELTGQINETVREAAERHDALFVLPDDADEHTSCAPPQQRWADIQGQQTDAYPLHPTSAGHEAMAAAVRDALGLEPVQP

SEQ ID NO: 15 - protein, 325 aa, Artificial Sequence
  Chemically synthesized amino acid sequence of CelA signal sequence-Cef II fusion protein
MGFGSAPIALCPLRTRRNALKRLLALLATGVSIVGLTALAGPPAQAREETAGAPPGESSGGIREEGAEASTSITDVYIALGDSYAAMGGRDQPLRGEPFCLRSSGNYPELLHAEVTDLTCQGAVTGDLLEPRTLGERTLPAQVDALTEDTTLVTLSIGGNDLGFGEVAGCIRERIAGENADDCVDLLGETIGEQLDQLPPQLDRVHEAIRDRAGDAQVVVTGYLPLVSAGDCPELGDVSEADRRWAVELTGQINETVREAAERHDALFVLPDDADEHTSCAPPQQRWADIQGQQTDAYPLHPTSAGHEAMAAAVRDALGLEPVQP

SEQ ID NO: 16 - DNA, 977 bp, Corynebacterium efficiens
atgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggcccgcgaggaaaccgccggcgcgccaccgggcgagtcgtcgggggaatccgagaagagggagctgaggcctccaccagcatcaccgacgtctacatcgccctcggcgattcgtacgccgcgatgggtgggcgcgaccagcccctgcgcggggagcccttctgcctccgcagttcgggtaactaccccgagctgcttcacgcggaggtgacggacctcacgtgccagggcgcggtcaccggcgacctgttggagccgcggactctgggcgagcgcaccctgccggcgcaggtggacgcgctgacggaggacaccacgctggtcaccctcagcatcggcgggaacgacctcggcttcggggaggtcgccggctgtatccgcgagcgcatcgccggcgagaacgcagatgactgcgtcgacctgctcggcgagaccatcggcgaacagctggaccagctcccgccccagctggaccgggtgcacgaggccatccgggaccgcgccggcgacgcgcaagtcgtggtcaccggttacctgccgctggtgtcagccggcgactgcccggaactcggcgacgtctccgaggccgacaggcgttgggccgtcgaactcaccggccagatcaacgagacagtacgggaggcggccgagcgccatgacgccctgttcgtgctgcccgacgacgccgacgagcacaccagctgcgcccccccgcagcagcggtgggcagacattcagggccagcagacggacgcctaccccctgcacccgacgtccgcgggccacgaagcaatggctgcggccgtccgggacgcgctgggactcgagccggtgcagccttga

SEQ ID NO: 17 - DNA, 33 bp, Artificial Sequence
  Synthetic primer
gcgatcctctagagatcgaacttcatgttcgag

SEQ ID NO: 18 - DNA, 35 bp, Artificial Sequence
  Synthetic primer
gcttataagcttcatcaggtggcggagttcaggac

SEQ ID NO: 19 - DNA, 48 bp, Artificial Sequence
  Synthetic primer
ggtggcctggatgcgcggggcgctggcctgtgccggggggccggctag

SEQ ID NO: 20 - DNA, 48 bp, Artificial Sequence
  Synthetic primer
ctagccggccccccggcacaggccagcgccccgcgcatccaggccacc

SEQ ID NO: 21 - DNA, 52 bp, Artificial Sequence
  Synthetic primer
agcgctagccggccccccggcacaggccgcgcccgcccaggccactccgacc

SEQ ID NO: 22 - DNA, 37 bp, Artificial Sequence
  Synthetic primer
tccggatccaggtcagtccaggccgaggacgtccatc

SEQ ID NO: 23 - DNA, 52 bp, Artificial Sequence
  Synthetic primer
agcgctagccggccccccggcacaggccgcccaacccgccgccgccgacggc

SEQ ID NO: 24 - DNA, 31 bp, Artificial Sequence
  Synthetic primer
tccggatccaggtcaggcggcgccgttgagg

SEQ ID NO: 25 - DNA, 861 bp, Artificial Sequence
  Chemically synthesized DNA sequence for the coding region of the SriII gene in plasmid Strep lipase B
atgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggccagcgccccgcgcatccaggccaccgactacgtggccctcggcgactcctactcctcgggcgtgggcgccggctcctacgacagctccagcggctcctgcaagcgctccaccaagtcctacccggccctgtgggccgcctcccacaccggcacccgcttcaacttcaccgcctgctccggcgcccgcaccggcgacgtcctggccaagcagctgaccccggtcaactccggcaccgacctggtctccatcaccatcggcggcaacgacgccggcttcgccgacaccatgaccacctgcaacctgcagggcgagtccgcctgcctggcccgcatcgccaaggcccgcgcctacatccagcagaccctgccggcccagctggaccaggtctacgacgccatcgactcccgcgccccggccgcgcaggtcgtcgtgctgggctacccgcgcttctacaagctgggcggctcctgcgccgtcggcctgtccgagaagtcccgcgccgccatcaacgccgccgccgacgacatcaacgccgtcaccgccaagcgcgccgccgaccacggcttcgccttcggcgacgtcaacaccaccttcgccggccacgagctgtgctcgggcgccccgtggctgcactccgtcaccctcccggtcgagaacagctaccacccgaccgccaacggccagtccaagggctacctgccggtcctgaactccgccacctga

SEQ ID NO: 26 - DNA, 933 bp, Artificial Sequence; identical to SEQ ID NO: 8
  Chemically synthesized DNA sequence encoding the S. coelicolor ScoIIA gene (in plasmid pDS104)

SEQ ID NO: 27 - DNA, 858 bp, Artificial Sequence; identical to SEQ ID NO: 12
  Chemically synthesized DNA sequence encoding the S. coelicolor ScoIIB gene (in plasmid pDS113)

SEQ ID NO: 28 - DNA, 977 bp, Artificial Sequence; identical to SEQ ID NO: 16
  Chemically synthesized DNA sequence for the Corynebacterium efficiens CefII gene sequence (as in plasmid pZQ201)

SEQ ID NO: 29 - DNA, 28 bp, Artificial Sequence
  Synthetic oligonucleotide
ccccggcacaggccagcgccccgcgcat

SEQ ID NO: 30 - DNA, 28 bp, Artificial Sequence
  Synthetic oligonucleotide
atgcgcggggcgctggcctgtgccgggg

(19) United States
(12) Patent Application Publication
(10) Pub. No.: US 2011/0281324 A1
(43) Pub. Date: Nov. 17, 2011

(54) Enzymes With Lipase Activity

(75) Inventors: Christian D. Adams, San Francisco, CA (US); Kai Bao, Palo Alto, CA (US); Katherine D. Collier, Los Altos, CA (US); Edwin Lee, San Francisco, CA (US); Andrei Miasnikov, Mountain View, CA (US); Zhen Qian, Shanghai (CN); Brian F. Schmidt, Half Moon Bay, CA (US); Danfeng Song, Shanghai (CN)

(73) Assignee: Danisco US Inc., Palo Alto, CA (US)

(21) Appl. No.: 13/132,252

(22) PCT Filed: Nov. 30, 2009
(86) PCT No.: PCT/US2009/066106

§ 371 Date: Jul. 25, 2011

Related U.S. Application Data
(60) Provisional application No. 61/118,852, filed on Dec. 1, 2008.

(51) Int. Cl. C11D 3/386; C12N 9/10; C12S 11/00; C12N 9/20

(52) U.S. Cl. 435/193; 435/198; 435/263; 510/320

(57) ABSTRACT

Described are detergent compositions comprising at least one lipase enzyme selected from SriII, ScoIIA, ScoIIB, CefII, and variants, thereof. The compositions are useful for removing oily stains from fabric.

### PRIORITY

The present application claims priority to U.S. Provisional Patent Application Ser. No. 61/118,852, filed on Dec. 1, 2008, which is hereby incorporated by reference.

### TECHNICAL FIELD

Described are detergent compositions comprising selected lipase enzymes. The compositions are useful for removing oily stains from fabrics.

### BACKGROUND

Removing oily soils (e.g., stains containing triglycerides and fatty acids) from fabric is a key unmet consumer need. Current market trends of (1) transition from powder to liquid detergents, (2) compaction of liquid detergents, (3) high efficiency washing machines, and (4) colder wash temperatures, provide both opportunities and challenges in meeting this need. Enzymes, such as lipases and acyltransferases, can bridge the performance gap between current technologies and next generation cold water and reduced surfactant laundry detergents. Lipases are enzymes capable of hydrolyzing lipids. They are used in a wide range of applications, such as processing of fats and oils, detergent compositions for cleaning purposes, and diagnostic reagents.

Because of the changing market trends, there is a need for new lipolytic enzymes with properties that are desirable for use in current laundry detergents and wash conditions.

### BRIEF SUMMARY OF THE INVENTION

In one aspect, the invention provides a polypeptide comprising, consisting of, or consisting essentially of the amino acid sequence depicted in SEQ ID NO: 2 (“SriII”) or a variant or homolog thereof, wherein the polypeptide has a lipase enzymatic activity and at least one additional activity selected from phospholipase, lysophospholipase, and acyltransferase. In some embodiments, the polypeptide also comprises a signal sequence as depicted in SEQ ID NOs: 1 or 3. In some embodiments, the polypeptide is a variant comprising at least about 60%, 65%, 70%, 75%, 80%, 85%, 90%, 95%, 97%, 98%, 99%, or 99.5% sequence identity to and having the enzymatic activities of SriII. In one embodiment, the polypeptide is stable to proteolysis, for example, stable to proteolysis by a subtilisin protease for at least 30 minutes at 30° C. The invention also provides a polynucleotide encoding the SriII polypeptide, such as a polynucleotide comprising, consisting of, or consisting essentially of the coding region of the polynucleotide sequence depicted in SEQ ID NOs: 4 or 25, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs: 4 or 25 and encoding a polynucleotide having the enzymatic activities of SriII. In some embodiments, the polynucleotide encoding the SriII polypeptide also encodes a signal sequence as depicted in SEQ ID NOs: 1 or 3. The invention also provides an expression vector comprising a polynucleotide encoding SriII, and a host cell comprising the expression vector.

In another aspect, the invention provides a polypeptide comprising, consisting of, or consisting essentially of the amino acid sequence depicted in SEQ ID NO: 6 (“ScoIIA”) or a variant or homolog thereof, wherein the polypeptide has a lipase enzymatic activity and at least one additional activity selected from phospholipase, lysophospholipase, and acyltransferase. In some embodiments, the polypeptide also comprises a signal sequence as depicted in SEQ ID NOs: 5 or 7. In some embodiments, the polypeptide is a variant comprising at least about 60%, 65%, 70%, 75%, 80%, 85%, 90%, 95%, 97%, 98%, 99%, or 99.5% sequence identity to and having the enzymatic activities of ScoIIA. The invention also provides a polynucleotide encoding the ScoIIA polypeptide, such as a polynucleotide comprising, consisting of, or consisting essentially of the coding region of the polynucleotide sequence depicted in SEQ ID NOs: 8 or 26, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs: 8 or 26 and encoding a polynucleotide having the enzymatic activities of ScoIIA. In some embodiments, the polynucleotide encoding the ScoIIA polypeptide also encodes a signal sequence as depicted in SEQ ID NOs: 5 or 7. The invention also provides an expression vector comprising a polynucleotide encoding ScoIIA, and a host cell comprising the expression vector.

In another aspect, the invention provides a polypeptide comprising, consisting of, or consisting essentially of the amino acid sequence depicted in SEQ ID NO: 10 (“ScoIIB”) or a variant or homolog thereof, wherein the polypeptide has a lipase enzymatic activity and at least one additional activity selected from phospholipase, lysophospholipase, and acyltransferase. In some embodiments, the polypeptide also comprises a signal sequence as depicted in SEQ ID NOs: 9 or 11. In some embodiments, the polypeptide is a variant comprising at least about 60%, 65%, 70%, 75%, 80%, 85%, 90%, 95%, 97%, 98%, 99%, or 99.5% sequence identity to and having the enzymatic activities of ScoIIB. The invention also provides a polynucleotide encoding the ScoIIB polypeptide, such as a polynucleotide comprising, consisting of, or consisting essentially of the coding region of the polynucleotide sequence depicted in SEQ ID NOs: 12 or 27, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs: 12 or 27 and encoding a polynucleotide having the enzymatic activities of ScoIIB. In some embodiments, the polynucleotide encoding the ScoIIB polypeptide also encodes a signal sequence as depicted in SEQ ID NOs: 9 or 11. The invention also provides an expression vector comprising a polynucleotide encoding ScoIIB, and a host cell comprising the expression vector.

In another aspect, the invention provides a polypeptide comprising, consisting of, or consisting essentially of the amino acid sequence depicted in SEQ ID NO: 14 (“CefII”) or a variant or homolog thereof, wherein the polypeptide has a lipase enzymatic activity. In some embodiments, the polypeptide or variant or homolog thereof has at least one additional enzymatic activity selected from phospholipase, lysophospholipase, and acyltransferase. In some embodiments, the polypeptide also comprises a signal sequence as depicted in SEQ ID NOs: 13 or 15. In some embodiments, the polypeptide is a variant comprising at least about 60%, 65%, 70%, 75%, 80%, 85%, 90%, 95%, 97%, 98%, 99%, or 99.5% sequence identity to and having the enzymatic activities of CefII. The invention also provides a polynucleotide encoding the CefII polypeptide, such as a polynucleotide comprising, consisting of, or consisting essentially of the coding region of the polynucleotide sequence depicted in SEQ ID NOs: 16 or 28, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs:16 or 28 and encoding a polynucleotide having the enzymatic activities of CefII. In some embodiments, the polynucleotide encoding the CefII polypeptide also encodes a signal sequence as depicted in SEQ ID NOs: 13 or 15. The invention also provides an expression vector comprising a polynucleotide encoding CefII, and a host cell comprising the expression vector.

In another aspect, the invention provides a detergent composition comprising, consisting of, or consisting essentially of, at least one polypeptide selected from the group consisting of SriII, ScoIIA, ScoIIB, CegII, and a variant, thereof, wherein the detergent composition exhibits improved cleaning of an oily stain compared to an equivalent detergent composition lacking the polypeptide. In some embodiments, the polypeptide is SriII. In some embodiments, the polypeptide is ScoIIA. In some embodiments, the polypeptide is ScoIIB. In some embodiments, the polypeptide is CefII.

In some embodiments, the polypeptide is SriII and the polypeptide comprises an amino acid sequence that is at least 90% identical to the amino acid sequence of SEQ ID NO: 2. In some embodiments, the polypeptide is ScoIIA and the polypeptide comprises an amino acid sequence that is at least 90% identical to the amino acid sequence of SEQ ID NO: 6. In some embodiments, the polypeptide is ScoIIB and the polypeptide comprises an amino acid sequence that is at least 90% identical to the amino acid sequence of SEQ ID NO: 10. In some embodiments, the polypeptide is CefII and the polypeptide comprises an amino acid sequence that is at least 90% identical to the amino acid sequence of SEQ ID NO: 14.

In a related aspect, the invention provides a detergent composition comprising a polypeptide having at least 90% amino acid sequence identity to a polypeptide comprising an amino acid sequence selected from the group consisting of SEQ ID NO: 2, SEQ ID NO: 6, SEQ ID NO: 10, SEQ ID NO: 14, wherein the detergent composition exhibits improved cleaning of an oily stain compared to an equivalent detergent composition lacking the polypeptide.

In some embodiments, the polypeptide has at least 90% amino acid sequence identity to a polypeptide comprising an amino acid sequence of SEQ ID NO: 2. In some embodiments, the polypeptide has at least 90% amino acid sequence identity to a polypeptide comprising an amino acid sequence of SEQ ID NO: 6. In some embodiments, the polypeptide has at least 90% amino acid sequence identity to a polypeptide comprising an amino acid sequence of SEQ ID NO: 10. In some embodiments, the polypeptide has at least 90% amino acid sequence identity to a polypeptide comprising an amino acid sequence of SEQ ID NO: 14.

In some embodiments, the polypeptide present in the detergent composition has lipase enzymatic activity and at least one additional activity selected from phospholipase, lysophospholipase, and acyltransferase activity. In some embodiments, the activity is phospholipase activity. In some embodiments, the activity is lysophospholipase activity. In some embodiments, the activity is acyltransferase activity. In some embodiments, the activity is a combination of these activities.

In some embodiments, the detergent composition comprises at lease one surfactant. In some embodiments, the detergent composition comprises at lease one additional polypeptide selected from the group consisting of a protease, an amylase, a cellulase, a laccase, a lipase, a phospholipase, a lysophospholipase, an acyltransferase, a perhydrolase, and an arylesterase.

In another aspect, the invention provides a method of cleaning an oily stain on a fabric, comprising contacting the stain with a detergent composition comprising at least one of SriII, ScoIIA, ScoIIB, and CefII polypeptide as described above under wash conditions in which the polypeptide is enzymatically active, wherein catalytic action of the polypeptide on a component of the stain facilitates removal of at least a portion of the stain from the fabric. In some embodiments, the oily stain comprises triglycerides.

In another aspect, the invention provides a method for assaying effectiveness of a composition (e.g., a detergent composition, a detergent composition comprising an enzyme, a buffer composition, a buffer composition comprising a surfactant and/or an enzyme) in removal of an oily stain from a fabric, comprising: (i) contacting a fabric swatch comprising an oily stain with the composition (in a well of a microtiter plate), (ii) mixing the composition and the stain-containing swatch, (iii) removing and rinsing the swatch, (iv) optionally adding the rinse to the wash liquor (supernatant) in the well of the microtiter plate, and (v) quantitating a component of the stain remaining on the cloth and released into the wash liquor, which optionally includes the rinse, or separately quantitating the stain component in the rinse and the wash liquor. In one embodiment, the detergent composition comprises an enzyme, such as a protease, amylase, cellulase, laccase, lipase, phospholipase, lysophospholipase, acyltransferase, perhydrolase, arylesterase, etc. In one embodiment, the stain comprises triglycerides, the detergent composition comprises an enzyme having lipase activity, and the fatty acids on the cloth, in the wash liquor, and in the rinse, or on the cloth and in the wash liquor to which the rinse has been added, are quantitated.

### BRIEF DESCRIPTION OF THE DRAWINGS

FIGS. 1A and 1B show schematic diagrams of the PCR reactions used to construct an A4 promoter-CelA signal-SriII fragment.

FIG. 2 shows a diagram of plasmid Strep lipase B used for the expression of SriII enzyme.

FIG. 3 shows a diagram of plasmid pDS104, used for the expression of ScoIIA enzyme.

FIG. 4 shows a diagram of plasmid pDS113, used for the expression of ScoIIB enzyme.

FIG. 5 shows a diagram of plasmid pZQ201 used for the expression of CefII enzyme.

FIG. 6 is a graph showing the stability of SriII lipase activity in the presence of protease (50 mM HEPES, pH 8.2, 30° C., 30 min).

FIG. 7 is a graph showing the hydrolysis of pNB by SriII lipase (25° C., 50 mM HEPES, pH 6.2, 2% PVA, 6 gpg).

FIG. 8 is a graph showing the hydrolysis of pNB by CefII lipase (25° C., 50 mM HEPES, pH 8.2, 2% PVA, 6 gpg).

FIG. 9 is a graph showing the hydrolysis of pNPP by SriII lipase (25° C., 50 mM HEPES, pH 6.2, 2% PVA, 6 gpg).

FIG. 10 is a graph showing the hydrolysis of pNPP by Sco II B lipase (25° C., 50 mM HEPES, pH 8.2, 2% PVA, 6 gpg).

FIG. 11 is a graph showing the hydrolysis of trioctanoate and tripalmitate by SriII (40° C., 60 mM, 50 mM HEPES, pH 8.2, 6 gpg, 2% gum arabic).

FIG. 12 is a graph showing L-alpha-phosphatidylcholine hydrolysis by SriII as assayed by detection of free fatty acids (40° C., 1.5 hr, 50 mM HEPES, pH 8.2, 2% PVA 6 gpg, NEFA).

FIG. 13 is a graph showing L-alpha-phosphatidylcholine hydrolysis by Sco IIB as assayed by detection of free fatty acids (40° C., 1.5 hr, 50 mM HEPES, pH 8.2, 2% PVA 6 gpg, NEFA).

FIG. 14 is a graph showing L-alpha-lysophosphatidylcholine hydrolysis by SriII as assayed by detection of free fatty acids (40° C., 16 hr, 50 mM HEPES, pH 8.2, 2% PVA 6 gpg, turbidity).

FIG. 15 is a graph showing hydrolysis of trioctanoic acid and triolein bound to cloth by ScoIIA (fatty acids produced at 40° C., 60 min, 50 mM HEPES, pH 8.2, 6 gpg, 0.98 mg/L Tide 2× Cold Water—inactivated).

FIG. 16 is a graph showing the stain removal performance of SriII on Sudan Red/lard microswatch in a 12-well plate assay (12-well swatch assay STC CFT CS-62 Lot 006 [Lard with Sudan Red] 50 mM HEPES, pH 8.2, 6 gpg, 20 min, 0.98 mg/L Tide).

FIG. 17 is a graph showing acyltransferase activity of SriII using 1,3-propanediol as acceptor assayed at 30° C., overnight in buffer.

### DETAILED DESCRIPTION

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); Hale and Marham, The Harper Collins Dictionary of Biology, Harper Perennial, NY (1991); and Kieser et al., Practical Streptomyces Genetics, The John Innes Foundation, Norwich, United Kingdom (2000) provide those of skill in the art with a general dictionaries of many of the terms used in the invention. Any methods and materials similar or equivalent to those described herein find use in the practice of the present invention, Accordingly, the terms defined immediately below are more fully described by reference to the Specification as a whole. Also, as used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context in which they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “enzyme” refers to any protein that catalyzes a chemical reaction. The catalytic function of an enzyme constitutes its “activity” or “enzymatic activity.” An enzyme typically is classified according to the type of catalytic function it carries out, e.g., hydrolysis of peptide bonds.

As used herein, the term “substrate” refers to a substance (e.g., a chemical compound) on which an enzyme performs its catalytic activity to generate a product.

As used herein, the term “acyl” refers to an organic group with the general formula RCO—, derived from an organic acid by removal of the —OH group. Typically, acyl group names end with the suffix “-oyl,” e.g., methanoyl chloride, CH₃CO—Cl, is the acyl chloride formed from methanoic acid, CH₃CO—OH).

As used herein, the term “acylation” refers to a chemical transformation in which one of the substituents of a molecule is substituted by an acyl group, or the process of introduction of an acyl group into a molecule.

As used herein, the term “transferase” refers to an enzyme that catalyzes the transfer of a functional group from one substrate to another substrate.

Related (and derivative) proteins encompass “variant” proteins. Variant proteins differ from a parent protein and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids.

In some embodiments, related proteins, such as variant proteins, comprise any of at least about 35%, 40%, 45%, 50%, 55%, 60%, 65%, 70%, 75%, 80%, 85%, 90%, 95%, 97%, 98%, 99%, or 99.5% amino acid sequence identity.

As used herein, the term “analogous sequence” refers to a polypeptide sequence within a protein that provides a similar function, tertiary structure, and/or conserved residues with respect to a reference protein. For example, in epitope regions that contain an alpha helix or a beta sheet structure, replacement amino acid(s) in an analogous sequence maintain the same structural element. In some embodiments, analogous sequences are provided that result in a variant enzyme exhibiting a similar or improved function with respect to the parent protein from which the variant is derived.

As used herein, “homologous protein” refers to a protein (e.g., a perhydrolase enzyme) that has similar function (e.g., enzymatic activity) and/or structure as a reference protein (e.g., a perhydrolase enzyme from a different source). Homologs may be from evolutionarily related or unrelated species. In some embodiments, a homolog has a quaternary, tertiary and/or primary structure similar to that of a reference protein, thereby potentially allowing for replacement of a segment or fragment in the reference protein with an analogous segment or fragment from the homolog, with reduced disruptiveness of structure and/or function of the reference protein in comparison with replacement of the segment or fragment with a sequence from a non-homologous protein.

As used herein, “wild-type,” “native,” and “naturally-occurring” proteins are those found in nature. The terms “wild-type sequence” refers to an amino acid or nucleic acid sequence that is found in nature or naturally occurring. In some embodiments, a wild-type sequence is the starting point of a protein engineering project, for example, production of variant proteins.

As used herein, “cleaning compositions” and “cleaning formulations” refer to compositions that find use in the removal of undesired compounds from items to be cleaned, such as fabric, dishes, contact lenses, other solid substrates, hair (shampoos), skin (soaps and creams), teeth (mouthwashes, toothpastes) etc. The term encompasses any materials/compounds selected for the particular type of cleaning composition desired and the form of the product (e.g., liquid, gel, granule, or spray composition), as long as the composition is compatible with the enzyme and other enzyme(s) used in the composition. The specific selection of cleaning composition materials are readily made by considering the surface, item or fabric to be cleaned, and the desired form of the composition for the cleaning conditions during use.

The terms further refer to any composition that is suited for cleaning, bleaching, disinfecting, and/or sterilizing any object and/or surface. It is intended that the terms include, but are not limited to detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish detergents).

Indeed, the term “cleaning composition” as used herein, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types.

The term “culturing” refers to growing a population of microbial cells under suitable conditions in a liquid or solid medium.

As used herein, the term “derivative” refers to a protein which is derived from a protein by addition of one or more amino acids to either or both the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is preferably achieved by modifying a DNA sequence which encodes for the native protein, transformation of that DNA sequence into a suitable host, and expression of the modified DNA sequence to form the derivative protein.

As used herein, the terms “detergent composition” and “detergent formulation” are used in reference to mixtures which are intended for use in a wash medium for the cleaning of soiled objects. In some preferred embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. Indeed, it is intended that in addition to enzyme, the term encompasses detergents that contain surfactants, transferase(s), hydrolytic enzymes, oxido reductases, builders, bleaching agents, bleach activators, bluing agents and fluorescent dyes, caking inhibitors, masking agents, enzyme activators, antioxidants, and solubilizers. DROPPS is a detergent composition having only a non-ionic ethoxylate surfactant and very low water content (about 10% by weight). In contrast, TIDE has both anionic and nonionic surfactants and higher water content (about 30-40% by weight).

As used herein, the phrase “detergent stability” refers to the stability of a detergent composition. In some embodiments, the stability is assessed during the use of the detergent, while in other embodiments, the term refers to the stability of a detergent composition during storage.

As used herein, “dishwashing composition” refers to all forms for compositions for cleaning dishes, including but not limited to granular and liquid forms.

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

As used herein, the term “expression” refers to the process by which a polypeptide is produced based on the nucleic acid sequence of a gene. The process includes both transcription and translation.

As used herein, “expression vector” refers to a DNA construct containing a DNA coding sequence (e.g., gene sequence) that is operably linked to one or more suitable control sequence(s) capable of effecting expression of the coding sequence in a host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. The plasmid is the most commonly used form of expression vector. However, the invention is intended to include such other forms of expression vectors that serve equivalent functions and which are, or become, known in the art.

As used herein, “fabric” encompasses any textile material. Thus, it is intended that the term encompass garments, as well as fabrics, yarns, fibers, non-woven materials, natural materials, synthetic materials, and any other textile material.

As used herein, “fabric cleaning composition” refers to all forms of detergent compositions for cleaning fabrics, including but not limited to, granular, liquid and bar forms.

As used herein, the term “host cell” refers to a cell or cell line into which a recombinant expression vector for production of a polypeptide may be transfected for expression of the polypeptide. Host cells include progeny of a single host cell, and the progeny may not necessarily be completely identical (in morphology or in total genomic DNA complement) to the original parent cell due to natural, accidental, or deliberate mutation. A host cell may be bacterial or fungal. A host cell includes cells transfected or transformed in vivo with an expression vector.

The term “introduced” in the context of inserting a nucleic acid sequence into a cell includes “transfection,” “transformation,” or “transduction” and refers to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed.

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide” and “nucleic acid” and “oligonucleotide” are used herein interchangeably. Polynucleotides of the invention may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions. Polynucleotide sequences are provided in the conventional 5′ to 3′ direction, unless otherwise specified.

As used herein, “polypeptide” refers to any composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptide sequences are provided in the conventional N to C direction, unless otherwise specified.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method. As with other polynucleotides, primer sequences are provided in the conventional 5′ to 3′ direction, unless otherwise specified.

The terms “recovered,” “isolated,” “purified,” and “separated” as used herein refer to a material (e.g., a protein, nucleic acid, or cell) that is removed from at least one component with which it is naturally associated. For example, these terms may refer to a material which is substantially or essentially free from components which normally accompany it as found in its native state, such as, for example, an intact biological system.

As used herein, the phrase, “stability to proteolysis” refers to the ability of a protein (e.g., an enzyme) to withstand proteolysis. It is not intended that the term be limited to the use of any particular protease to assess the stability of a protein.

A “lipolytic enzyme” (E.C. 3.1.1), as used herein, refers to any acyl-glycerol carboxylic ester hydrolase. “Lipolytic enzymes” include lipases (triacylglycerol acylhydrolases, E.C. 3.1.1.3) or cutinases (E.C. 3.1.1.50). Lipase has higher selectivity toward long chain triglycerides contained in fat than cutinase. Cutinase has higher selectivity toward short chain triglycerides contained in fat than lipase.

SriII

The invention provides a polypeptide designated “SriII” herein comprising, consisting of, or consisting essentially of the amino acid sequence depicted in SEQ ID NO: 2 or a variant or homolog thereof, wherein the polypeptide has a lipase enzymatic activity and at least one additional activity selected from phospholipase, lysophospholipase, and acyltransferase. The SriII polypeptide is stable to proteolysis, for example, stable to proteolysis by a subtilisin protease for at least 30 minutes at 30° C. The invention also provides a polynucleotide encoding the SriII protein, such as a polynucleotide comprising, consisting of, or consisting essentially of the coding region of the polynucleotide sequence depicted in SEQ ID NOs: 4 or 25, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs: 4 or 25 and encoding a polynucleotide having the enzymatic activities of SriII. The invention also provides an expression vector comprising a polynucleotide encoding SriII, and a host cell comprising the expression vector.

The SriII polypeptide, or variant or homolog thereof, may be used in an application in which the enzymatic activities demonstrated for this polypeptide herein, are useful, such as, for example, a method for cleaning an oily stain, a food processing method, a method for degumming of edible oils, a method for synthesis of a flavor, a method for synthesis of a surfactant, a waste treatment method, or a method for generation of an emulsifier (e.g., for baking applications).

ScoIIA

The invention provides a polypeptide designated “ScoIIA” herein comprising, consisting of, or consisting essentially of the amino acid sequence depicted in SEQ ID NO: 6 or a variant or homolog thereof, wherein the polypeptide has a lipase enzymatic activity and at least one additional activity selected from phospholipase, lysophospholipase, and acyltransferase. The invention also provides a polynucleotide encoding the ScoIIA protein, such as a polynucleotide comprising, consisting of, or consisting essentially of the coding region of the polynucleotide sequence depicted in SEQ ID NO: 8 or 26, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs: 8 or 26 and encoding a polynucleotide having the enzymatic activities of ScoIIA. The invention also provides an expression vector comprising a polynucleotide encoding ScoIIA, and a host cell comprising the expression vector.

The ScoIIA polypeptide, or variant or homolog thereof, may be used in an application in which the enzymatic activities demonstrated for this polypeptide herein, are useful, such as, for example, a method for cleaning an oily stain, a food processing method, a method for degumming of edible oils, a method for synthesis of a flavor, a method for synthesis of a surfactant, a waste treatment method, or a method for generation of an emulsifier (e.g., for baking applications).

ScoIIB

The invention provides a polypeptide designated “ScoIIB” herein comprising, consisting of, or consisting essentially of the amino acid sequence depicted in SEQ ID NO: 10 or a variant or homolog thereof, wherein the polypeptide has a lipase enzymatic activity and at least one additional activity selected from phospholipase, lysophospholipase, and acyltransferase. The invention also provides a polynucleotide encoding the ScoIIB protein, such as a polynucleotide comprising, consisting of, or consisting essentially of the coding region of the polynucleotide sequence depicted in SEQ ID NOs: 12 or 27, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs: 12 or 27 and encoding a polynucleotide having the enzymatic activities of ScoIIB. The invention also provides an expression vector comprising a polynucleotide encoding ScoIIB, and a host cell comprising the expression vector.

The ScoIIB polypeptide, or variant or homolog thereof, may be used in an application in which the enzymatic activities demonstrated for this polypeptide herein, are useful, such as, for example, a method for cleaning an oily stain, a food processing method, a method for degumming of edible oils, a method for synthesis of a flavor, a method for synthesis of a surfactant, a waste treatment method, or a method for generation of an emulsifier (e.g., for baking applications).

CefII

The invention provides a polypeptide designated “CefII” herein comprising, consisting of, or consisting essentially of the amino acid sequence depicted in SEQ ID NO: 14 minus the signal sequence or a variant or homolog thereof, wherein the polypeptide has a lipase enzymatic activity. In some embodiments, the polypeptide or variant or homolog thereof has at least one additional enzymatic activity selected from phospholipase, lysophospholipase, and acyltransferase. The invention also provides a polynucleotide encoding the CefII protein, such as a polynucleotide comprising, consisting of, or consisting essentially of the coding region of the polynucleotide sequence depicted in SEQ ID NOs: 16 or 28, or a polynucleotide comprising at least about 60%, 65%, 70%, 75%, 80%, 85%, 90%, 95%, 97%, 98%, 99%, or 99.5% sequence identity to the coding region of the polynucleotide sequence depicted in SEQ ID NOs:16 or 28 and encoding a polynucleotide having the enzymatic activities of CefII. The invention also provides an expression vector comprising a polynucleotide encoding CefII, and a host cell comprising the expression vector.

The CefII polypeptide, or variant or homolog thereof, may be used in an application in which the enzymatic activities demonstrated for this polypeptide herein, are useful, such as, for example, a method for cleaning an oily stain, a food processing method, a method for degumming of edible oils, a method for synthesis of a flavor, a method for synthesis of a surfactant, a waste treatment method, or a method for generation of an emulsifier (e.g., for baking applications).

Detergent Compositions

The invention provides a detergent composition comprising at least one of the enzymes described herein (i.e., SriII, ScoIIA, ScoIIB, CefII, or a variant or homolog thereof). The detergent contains an enzyme described herein and one or more components of a detergent composition such as surfactants, hydrolytic enzymes, builders, bleaching agents, bleach activators, bluing agents, fluorescent dyes, caking inhibitors, masking agents, antioxidants, and solubilizers.

In one embodiment, the invention provides a solid detergent composition comprising one or more of SriII, ScoIIA, ScoIIB, CefII, or a variant or homolog thereof. The solid detergent composition is often in a dry powder and/or granular form. The amount of the enzyme in the solid detergent is generally 0.001-1%, preferably 0.01-0.5%, and more preferably 0.05-0.2% (w/w).

In a further embodiment, the invention provides a liquid detergent composition comprising one or more of SriII, ScoIIA, ScoIIB, CefII, or a variant or homolog thereof. In the liquid detergent composition, the enzyme is in an amount of 0.001-1%, preferably 0.01-0.5%, and more preferably 0.05-0.2% (w/v). The liquid detergent composition is in general diluted 200-5,000 fold, preferably 500-2,000 fold, and more preferably about 1000 fold, to prepare a washing solution in a washing machine.

Methods of Cleaning

The invention provides a method of cleaning a stain, for example, an oily stain on a fabric, comprising contacting the stain with at least one enzyme described herein (SriII, ScoIIA, ScoIIB, and/or CefII, or a variant or homolog thereof), wherein catalytic action of the enzyme on a component of the stain is effective to remove at least a portion of that component from the stain.

In some embodiments, the method comprises contacting the stain with a detergent composition as described above comprising at least one of the enzymes described herein (i.e., SriII, ScoIIA, ScoIIB, CefII, or a variant or homolog thereof). The detergent composition may be diluted to provide a wash solution, e.g., a wash solution in a laundry machine. The washing solution has a pH range generally 4-11, preferably 5-10, and more preferably 8-9. The treatment temperature is in general 15-60° C., preferably 20-50° C., and more preferably 30-40° C. The concentration of the enzyme in the washing solution is generally 0.01-10 mg/L, preferably 0.1-5 mg/L, and more preferably 0.5-2 mg/L. In some embodiments, the enzyme degrades triglycerides in the oily stain, which facilitates removal of the stain.

In some embodiments, the invention provides a prespotting composition for use in a method of pretreating an oily stain on a fabric. The prespotting composition may contain at least one of the enzymes described herein ((i.e., SriII, ScoIIA, ScoIIB, CefII, or a variant or homolog thereof) in an amount of 0.001-1%, preferably 0.01-0.5%, and more preferably 0.05-0.2% (w/v) and at a pH range generally 4-11, preferably 5-10, and more preferably 5-7. The prespotting composition is applied to the stain prior to laundering.

In some embodiments, the invention provides a composition for removal of an oily stain or residue from a hard surface. The hard surface cleaning composition may contain at least one of the enzymes described herein ((i.e., SriII, ScoIIA, ScoIIB, CefII, or a variant or homolog thereof) in an amount of 0.001-1%, preferably 0.01-0.5%, and more preferably 0.05-0.2% (w/v) and at a pH range generally 4-11, preferably 5-10, and more preferably 5-7. The hard surface cleaning composition is applied to the oily stain or residue and then washed or wiped away to remove at least a portion of the stain or residue, or a component thereof, from the surface.

Assay Procedure for Determining Effectiveness of Oily Stain Removal from Fabric

The invention provides an assay method for determining the extent of removal of an oily stain from a fabric, comprising: (i) contacting a fabric swatch comprising an oily stain with a composition to be assayed for effectiveness in oily stain removal (e.g., a detergent composition, a detergent composition comprising an enzyme, a buffer composition, a buffer composition comprising a surfactant and/or an enzyme) in a well of a microtiter plate (e.g., 6 well, 12 well, 48 well, 96 well, etc.), (ii) mixing the composition and the stain-containing swatch, (iii) removing and rinsing the swatch, (iv) optionally adding the rinse to the wash liquor (supernatant) in the well of the microtiter plate, and (v) quantitating a component of the stain remaining on the cloth and released into the wash liquor (optionally including the rinse or quantitated separately from the rinse). In one embodiment, the detergent composition comprises an enzyme, such as a protease, amylase, cellulase, laccase, lipase, phospholipase, lysophospholipase, acyltransferase, perhydrolase, arylesterase, etc.

In one embodiment, the stain comprises triglycerides, the detergent composition comprises an enzyme having lipase activity, and the fatty acids on the cloth, in the wash liquor, and in the rinse, or on the cloth and in the wash liquor to which the rinse has been added, are quantitated.

In one example of the assay method, the assay is performed as follows: A cotton swatch is placed in a well of a 96 well microtiter plate. 1 μl of triglyceride is dotted onto the swatch. Liquid detergent containing a lipase enzyme is added to the well. The microtiter plate is shaken, for example, at 20, 25, 30, 35, or 40° C. for about 20 minutes, 30 minutes, 40 minutes, 1 hour, 2 hours, 5 hours, 10 hours, 15 hours, or 20 hours. The swatch is removed from the well and rinsed. The rinse is optionally added to the wash liquor remaining in the well, or assayed separately from the wash liquor remaining in the well. Both the swatch and the wash liquor (optionally containing the rinse) are assayed for the presence of free fatty acids, and optionally the rinse is separately assayed for the presence of free fatty acids. One example of an assay procedure for quantitating free fatty acids is the NEFA assay, which measures fatty acids produced from hydrolysis of triglycerides on fabric. (NEFA Assay Kit, Wako Diagnostics, Richmond, Va.; Hoffmann et al. (1986) Clinical Chemistry 32(3): 545-547.) The assay measures the fatty acids which have been released into solution, as well as those remaining on the fabric.

The following examples are intended to illustrate, but not limit, the invention.

### EXAMPLES

### Example 1

### General Assay Procedures

In the following Examples, various assays were used as set forth below for ease in reading. Any deviations from the protocols provided below are indicated in the Examples.

A. Para-Nitrophenyl Butyrate Ester (pNB) Assay to Determine Lipase/Esterase Activity

Equipment: Specrophotometer capable of kinetic measurements and temperature control; water bath at 25° C.; and 96-well microtiter plates.

Materials: Assay buffer: 50 mM HEPES pH 8.2, 6 gpg, 3:1 Ca:Mg hardness, 2% polyvinyl alcohol (PVA) (Sigma); and Substrate: 20 mM p-nitrophenyl butyrate (pNB; Sigma, CAS 2635-84-9, catalog number N9876) dissolved in DMSO (Pierce, 20688, water content <0.2%), stored at −80° C. for long term storage.

Procedure: Serial dilutions of enzyme samples in assay buffer were prepared in 96-well microtiter plates and equilibrated at 25° C. 100 μL of 1:20 diluted substrate (in assay buffer) was added to another microtiter plate. The plate was equilibrated to 25° C. for 10 minutes with shaking at 300 rpm. 10 μL of enzyme solution from the dilution plate was added to the substrate containing plate to initiate the reaction. The plate was immediately transferred to a plate reading spectrophotometer thermostated at 25° C. The absorbance change in kinetic mode was read for 5 minutes at 410 nm. The background rate (with no enzyme) was subtracted from the rate of the test samples.

B. Para-Nitrophenyl Palmitate (pNPP) Assay to Determine Lipase/Esterase Activity

The pNPP assay to measure lipase/esterase activity was performed exactly as described in the pNB assay except that the substrate used was 20 mM p-nitrophenyl Palmitate (pNPP; Sigma, CAS1492-30-4, catalog number N2752) dissolved in DMSO (Pierce, 20688, Water content <0.2%), stored at −80° C. for long term storage.

### C. Triglyceride Hydrolysis Assay in 96-Well Microtiter Plates

This assay was designed to measure enzymatic release of fatty acids from triglyceride substrate. The assay consists of a hydrolysis reaction where incubation of enzyme with a triglyceride emulsion results in liberation of fatty acids, detection of the liberated fatty acids and measurement in the reduction of turbidity of the emulsified substrate.

Equipment: Plate Reading Spectrophotometer capable of end point measurements (SpectraMax Plus384 (Molecular Devices, Sunnyvale, Calif.); 96-well microtiter plates, and an Eppendorf Thermomixer.

Triglyceride substrates: Glycerol trioctanoate (Sigma, CAS 538-23-8, catalog number T9126-100ML); Glyceryl trioleate (Fluka, CAS122-32-7, catalog number 92859); and Glyceryl tripalmitate (Fluka, CAS 555-44-2, catalog number 92902).

Reagents: NEFA (non-esterified fatty acid) assay reagent (HR Series NEFA-HR (2) NEFA kit, WAKO Diagnostics, Richmond, Va.).

Procedure: Emulsified triglycerides (0.75% (v/v or w/v)) were prepared by mixing 50 ml of gum arabic (Sigma, CAS 9000-01-5, catalog number G9752; 10 mg/ml gum arabic solution made in 50 mM MOPS pH 8.2), 6 gpg water hardness, in 50 mM HEPES, pH 8.2) with 375 μlof triglyceride (if liquid) or 0.375 g triglyceride (if solid). The solutions were mixed and sonicated for at least 2 minutes to prepare a stable emulsion.

200 μL of emulsified substrate was added to a 96-well microtiter plate. Twenty microliters of serially diluted enzyme samples were added to the substrate containing plate. The plate was covered with a plate sealer and incubated at 40° C. shaking for 1-2 hours. After incubation, the presence of fatty acids in solution was detected using the HR Series NEFA-HR (2) NEFA kit as indicated by the manufacturer. The NEFA kit measures non-esterified fatty acids.

### D. Assay to Detect Lysophospholipase Activity in 96-Well Microtiter Plates

Detection of lysophospholipase activity was performed as described in “Triglyceride hydrolysis assay to determine lipase activity in 96-well microtiter plates” except using L-α-lysophosphatidylcholine (Sigma L0906-500 mg) as substrate.

### E. Assay to Detect Phospholipase Activity in 96-Well Microtiter Plates

Phospholipase activity was measured as described “Triglyceride hydrolysis assay to determine lipase activity in 96-well microtiter plates” except using L-α-phosphatidylcholine (Sigma P5394) as substrate.

### F. Terg-O-Tometer Application for Cleaning Performance Determination

A protocol for assessing lipid soil cleaning was performed whereby the soil level on a fabric swatch was measured before and after cleaning. The fabric swatches consisted of Pigment Vegetable Oil stain (WFK10 PF) and Pigment-Oil-Milk (CFT AS-10) and were purchased from Test Fabrics, Inc. (West Pittiston, Pa.). Each stain was measured before and after treatment by optical reflectance using a Minolta Reflectometer CR-410 set to a D65 (6500° K) standard illuminant. The difference in the L, a, and b values was converted to total color difference (dE), as defined by the CIE-LAB color space. Cleaning of the stains was expressed as percent stain removal index (% SRI) by taking a ratio between the color difference before and after washing and comparing it to the difference of unwashed soils (before wash) to unsoiled fabric.

Cleaning experiments were conducted in a Terg-o-tometer (United States Testing Co., Hoboken, N.J., USA) equipped with 6 stainless steel 2 L pots fitted with overhead agitators. Each treatment was conducted in 1 L total volume containing 6 grains per gallon 3:1 (Calcium:Magnesium) water hardness. Detergent used in the wash experiment was heat inactivated commercially available Tide Cold Water 2× Laundry Detergent (Procter and Gamble, Cincinnati, Ohio, purchased from a local supermarket store).

Heat inactivation of commercial detergent formulas serves to destroy the enzymatic activity of enzyme components while retaining the properties of non-enzyme components. Heat inactivation was performed by placing pre-weighed liquid detergent (in glass bottle) in a water bath at 95° C. for 8 hours. For testing of enzyme activity in heat-inactivated detergents, working solutions of detergents were made from the heat inactivated stock solution in buffer (6 gpg of water hardness, 50 mM HEPES, pH 8.2).

### G. Triglyceride Hydrolysis Assay on Microswatches to Determine Lipase Activity

Microswatches treated with triglycerides were prepared as follows. EMPA 221 unsoiled cotton fabrics (Test Fabrics Inc. West Pittiston, Pa.) were cut to fit 96-well microtiter plates. 0.5-1 μl of neat triolein, trioctanoate, or triplamitin were spotted on the microswatches. The swatches were left at room temperature for about 10 minutes. One triglyceride treated microswatch was placed in each well of a microtiter plate. 150 μl of heat inactivated Tide Cold Water 2× Laundry Detergent (prepared as described in “Terg-o-tometer application for cleaning performance determination”) was added to each well containing a microswatch. 10 μL of serially diluted enzyme samples were added to these wells. The plate was sealed with a plate sealer and incubated at 750 rpm at 40° C. for 60 minutes. After incubation, the supernatant was removed (and saved) from the swatches and the swatches were rinsed with 100 μL of detergent (save rinse) and blotted dry on paper towels. The presence of fatty acids in solution (supernatant and rinse) and remaining on the cloth was detected using the HR Series NEFA-HR (2) NEFA kit (WAKO Diagnostics, Richmond, Va.) as indicated by the manufacturer.

### Example 2

### Cloning and Expression of Streptomyces rimosus Enzyme with Lipase Activity (“SriII”)

Generation of a synthetic gene encoding the SriII enzyme (Swissprot: Q93MW7, Pubmed: AAK84028.1, mature protein sequence as depicted in SEQ ID NO. 2) was performed based on a codon selection method for improving expression in Streptomyces lividans. Plasmid pKB105 was used for the expression of SriII gene in Streptomyces lividans.

PCR reactions were performed using the SriII synthesized gene sequence and plasmid pKB105 (described in U.S. Publication No. 2006/0154843) as the source of the A4 promoter-CelA signal sequence.

Primers for splice-overlap extension PCR were synthesized with overlapping regions between the A4 promoter-CelA signal sequence and the SriII encoding gene sequence. The outside primers were engineered with restriction sites for cloning into plasmid pKB105 (Table 1).

TABLE 1
Primer sequences used for splice-overlap extension
PCR

| SEQ | | |
|---|---|---|
| ID | | |
| NO. | Primer | Sequence |
| 17 | EL-917 (+) | GCGATCCTCTAGAGATCGAACTTCATGTTCGAG |
| 18 | EL-920 (−) | GCTTATAAGCTTCATCAGGTGGCGGAGTTCAGGAC |
| 19 | EL-921 (−) | GGTGGCCTGGATGCGCGGGGCGCTGGCCTGTGCCG |
| | | GGGGGCCGGCTAG |
| 20 | EL-922 (+) | CTAGCCGGCCCCCCGGCACAGGCCAGCGCCCCGC |
| | | GCATCCAGGCCACC |

Two separate PCR reactions were performed to amplify the A4 promoter-CelA and SriII gene fragments. PCR tube 1 contained 1 μl plasmid pKB105 (25 ng/μl) as template to amplify the A4 promoter-CelA signal sequence, 0.5 μl of primer E-917 (25 mM), 0.5 μl of primer EL-921 (25 mM), 0.5 μl dNTP (25 mM), 10 μl 5× Herculase II Fusion Buffer (Stratagene, La Jolla, Calif.), 0.5 μl Herculase II Fusion DNA Polymerase (Stratagene), and 37 μl deionized water. PCR tube 2 contained 1 μl of the synthetic SriII gene in shuttle vector (25 ng/nμl) as template to amplify the SriII gene, 0.5 μl of primer EL-920 (25 mM), 0.5 μl of primer EL-922 (25 mM), 0.5 μl dNTP (25 mM), 10 μl 5× Herculase II Fusion Buffer (Stratagene), 0.5 μl Herculase II Fusion DNA polymerase (Stratagene), and 37 μl deionized water.

PCR was performed using a MJ Research PTC-200 Peltier Thermal Cycler (Bio-Rad Laboratories, Hercules, Calif.) with the following conditions to amplify both fragments as follows: 95° C. for 2 minutes (first cycle only), 95° C. for 25 seconds, 60° C. for 25 seconds, 72° C. for 25 seconds, 27 cycles with extension at 72° C. for 3 minutes.

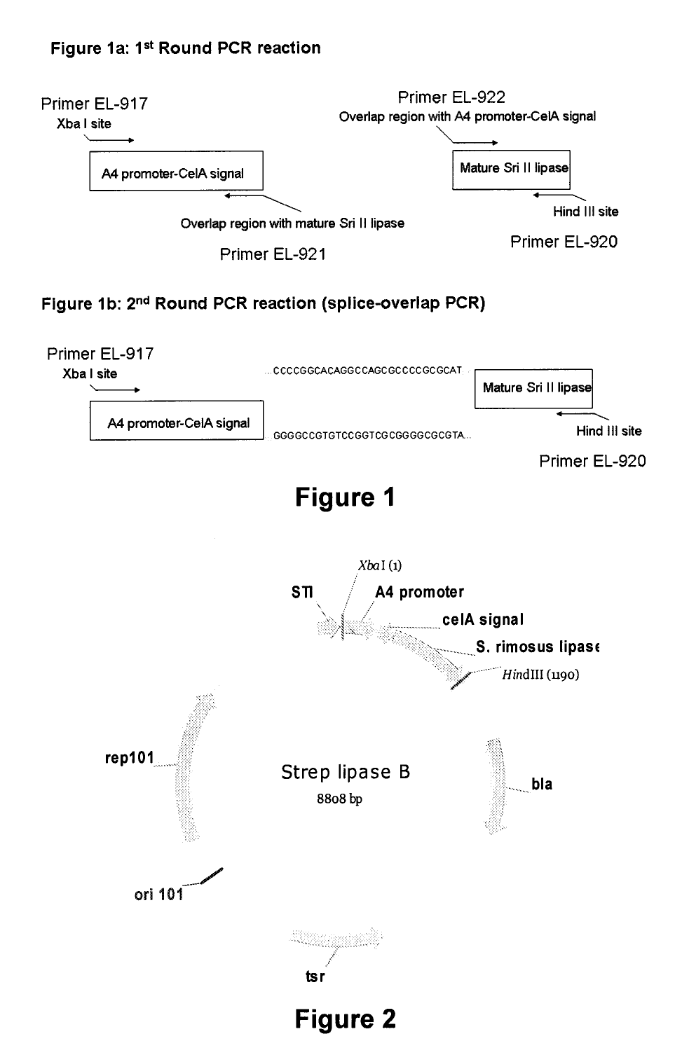

First round PCR resulted in the products shown in FIG. 1A, which had the indicated sizes: A4 promoter-CelA fragment (463 bp); SriII gene fragment (726 bp). PCR products were purified using QIAquick PCR Purification Kit (Qiagen Inc., Valencia, Calif.). Splice-overlap extension PCR was performed by combining the A4 promoter-CelA fragment and the SriII gene fragment. The splice-overlap extension PCR reaction contained 1 μl of A4 promoter-CelA fragment, 1 μl SriII gene fragment, 0.5 μl Primer EL-917 (25 mM), 0.5 μl Primer EL-920 (25 mM), 10 μl 5× Herculase II Fusion Buffer, 0.5 μl dNTP (25 mM), 0.5 μl Herculase II Fusion DNA Polymerase (Stratagene), and 36 μl deionized water. PCR conditions were as follows: 95° C. for 2 minutes (first cycle only), 95° C. for 25 seconds, 60° C. for 25 seconds, 72° C. for 33 seconds, 27 cycles with extension at 72° C. for 3 minutes.

Splice-overlap extension PCR resulted in the products shown in FIG. 1B, which had the indicated sizes: SriII PCR fragment: ˜1189 bp. Splice-overlap extension PCR product was separated by electrophoresis using 1.2% E-gels (Invitrogen Corp., Carlsbad, Calif.) and purified using the QIAquick Gel Extraction Kit (Qiagen Inc., Valencia, Calif.). The splice-overlap extension PCR product was then digested with Hind III and Xba I at 37° C. for 3 hours in a reaction containing 6 μl PCR product, 4 μl 10× Roche Buffer B (Roche, Indianapolis, Ind.), 1.5 μl Hind III (10 U/μl, Roche), 1.5 μl XbaI (10 U/μl, Roche), and 27 μl deionized water.

About 500 ng of plasmid DNA pKB105 (2.5 μl pKB105 (201 ng/μl) was also digested with Hind III and Xba I at 37° C. for 3 hours in a reaction containing 4 μl 10× Roche Buffer B, 1.5 μl Hind III (10 U/μl), 1.5 μl XbaI (10 U/μl), and 30.5 μl deionized water.

The digested plasmid pKB105 DNA and splice-overlap extension PCR fragments were separated on a 1.2% E-gel and purified using the QIAquick Gel Extraction Kits. 2 μl digested purified plasmid pKB105 DNA and 5 μl splice-overlap extension PCR fragments were ligated overnight at 16° C. in a reaction containing 2 μl 10× T4 DNA ligase buffer (New England Biolabs, Ipswich, Mass.), 1 μl T4 DNA ligase (400 U/μl, New England Biolabs), and 10 μl deionized water.

The next day, chemically competent TOP10 E. coli cells (Invitrogen Corp., Carlsbad, Calif.) were transformed with 2 μl of the overnight ligation reaction following the manufacturer's protocol. Transformed cells were plated on Luria Agar plates supplemented with 50 μg/μl carbenicillin and incubated overnight at 37° C. The next day, 5 transformants were picked and inoculated in 5 ml of Luria broth supplemented with 50 μg/μl carbenicillin. Cultures were grown overnight at 37° C. The next day, plasmid DNA was extracted using QIAquick Spin Miniprep Kit (Qiagen Inc.). Independent clones showing the expected plasmid backbone and insert sizes were subjected to confirmatory DNA sequencing. When translated, the DNA sequences for the PCR amplified SriII coding regions of the genes were 100% identical to those previously reported (Protein ID: Q93MW7).

Following DNA sequencing confirmation, the plasmid Strep lipase B (depicted in FIG. 2) was used to transform S. lividans TK23 derivative protoplasts (described in U.S. Publication No. 2006/0154843). The DNA sequence for the coding region of SriII is shown as SEQ ID NO: 25. The coding sequence of the S. rimosus gene is shown in bold. The coding sequence for the CelA signal sequence is shown in normal typeface.

Transformation of Streptomyces lividans and Expression of SriII Protein

The host Streptomyces lividans TK23 derivative strain was transformed with Strep lipase B plasmid. Transformation was performed according to the protoplast method described in Kieser et al., Practical Streptomyces Genetics, The John Innes Foundation, Norwich, United Kingdom (2000). Transformed cells were plated on R5 selection plates and incubated at 30° C. for 3 days. One clone from the Streptomyces transformation plate was inoculated in TSG medium in shake flasks at 28° C. for 3 days. Cultures were then transferred to a Streptomyces 2 Modified Medium and incubated for an additional 4 days at 28° C.

TSG medium: 16 g BD Difco Tryptone, 4 g BD Bacto soytone, 20 g Sigma Caseine (hydrolysate), and 10 g Potassium Phosphate, Dibasic brought to 1 liter. After autoclaving, 50% Glucose was added to a final concentration of 1.5%

Streptomyces 2 Modified Medium: 2.4 g Citric Acid Monohydrate, 6 g Biospringer Yeast Extract, 2.4 g Ammonium Sulfate, 2.4 g Magnesium Sulfate Heptahydrate, 0.5 ml Mazu DF204 (antifoam), 5 ml Streptomyces modified trace elements (1 liter stock solution contains 250 g citric acid monohydrate, 3.25 g FeSO₄.*7H₂O; 5 g ZnSO₄*7 H₂O, 5 g MnSO₄*H₂O, 0.25 g H₃BO₃). Adjust pH to 6.9. After autoclaving, add 2 ml 100 mg/ml calcium chloride, 200 ml 13% (w/v) potassium phosphate, monobasic (pH 6.9), and 20 ml 50% glucose.

R5 plates: 206 g sucrose, 0.5 g K₂SO₄, 20.24 g MgCl₂, 20 g glucose, 0.2 g Difco casamino acids, 10 g Difco yeast extracts and 11.46 g TES, 4 g L-Asp, 4 ml of trace elements and 44 g Difco agar, 20 ml 5% K₂HPO₄ and 8 ml 5M CaCl₂′ 2H₂O and 14 ml 1N NaOH were added to a final volume of 1 liter after autoclaving. After 20 hours a layer of thiostrepton (50 μg/ml final concentration) was plated on the top of the plates.

### Purification of SriII Enzyme

Streptomyces lividans cells expressing SriII protein were grown at 14 L fermentor scale under typical fermentation conditions as outlined in Kieser et al., Practical Streptomyces Genetics, The John Innes Foundation, Norwich, United Kingdom (2000), and media components listed in Streptomyces 2 modified media (see above). Ultra-filtered concentrate (UFC) from the 14 L fermentor scale was diluted 5-fold with 50 mM Tris-HCl pH 8.0 buffer and ammonium sulfate was added to a final concentration of 1 M. Enzyme purification from the culture broth was done on a high density, FastFlow Phenyl Sepharose resin column equilibrated with 1 M ammonium sulfate in 50 mM Tris-HCl pH 8.0 buffer. Sample was loaded at ½ the flow rate of the equilibration flow rate (12 ml/min) and washed after loading. A gradient was used to reduce the concentration of the 1 M ammonium sulfate to 0 M. Contaminant proteins were washed off the column with the 50 mM Tris pH 8.0 buffer. SriII enzyme was eluted with a buffer containing 50 mM Tris HCl pH 8.0 and 40% propylene glycol. Fractions were assayed using the pNB assay and those containing lipase activity were pooled and concentrated for subsequent use.

### Example 3

### Cloning and Expression of Enzymes with Lipase Activity from Streptomyces coelicolor M145 (“ScoIIA” and “ScoIIB”)

The genes encoding lipases ScoIIA (NCBI: NP_{—}631558, CAC42140, SEQ ID NO: 5) and ScoIIB (NCBI: NP_{—}625998, CAB50940, SEQ ID NO: 9) were isolated from genomic DNA of Streptomyces coelicolor strain M145, a prototrophic derivative of strain A3(2) lacking its two plasmids (SCP1 and SCP2).

Mycelia preparation and genomic DNA isolation was performed as described in Kieser et al., Practical Streptomyces Genetics, The John Innes Foundation, Norwich, United Kingdom (2000).

For PCR of the ScoIIA gene, the following primers were used:

| | |
|---|---|
| Primer 1 (Sdf5): 5-agcgctagccggccccccggcacaggccgcgcccgcccaggccactccgacc-3 | (SEQ ID NO: 21) |
| Primer 2 (Sdf6): 5-tccggatccagg tcagtccaggccgaggacgtccatc-3 | (SEQ ID NO: 22) |

For PCR of the ScoIIB gene, the following primers were used:

| | |
|---|---|
| Primer 1 (1725-Fw): 5′-agcgctagccggccccccggcacaggccgcc caacccgccg ccgccgacgg c-3′ | (SEQ ID NO: 23) |
| Primer 2 (1725-Rv): 5′-tccggatccaggtca ggcggcgccgttgagg-3′ | (SEQ ID NO: 24) |

PCR reactions were performed using the extracted genomic DNA as the template in order to amplify the desired genes for ScoIIA and ScoIIB. Primers were designed with engineered restriction sites for cloning into vector pKB105 (described in U.S. Publication No. 2006/0154843). PCR was performed on a MJ Research PTC-200 Peltier Thermal Cycler (Bio-Rad Laboratories) using KOD Hot Start Master Mix Kit (Cat. # 71842, Novagen, Gibbstown, N.J.) as described by the manufacturer.

Two PCR products were produced with the following sizes:

ScoIIA fragment: 972 bp, NheI site+C-terminal of celA signal sequence+ScoIIA+BamHI site. ScoIIB fragment: 720 bp, NheI site+C-terminal of celA signal sequence+ScoIIB+BamHI site.

PCR products were isolated by electrophoresis using 1.2% E-gels (Invitrogen Corp.) and purified using the QIAquick Gel Extraction Kit (Qiagen Inc.). The DNA was digested with NheI and BamHI restriction endonucleases (New England Biolabs) as follows: 6 μl DNA (100 ng/μl, 4 μl 10×NEB Buffer 2 (New England Biolabs B7002S), 1.5 μl NheI (10 U/μl), 1.5 μl BamHI (20 U/μl), and 27 μl autoclaved, deionized water, incubated at 37° C. for 3 hours. Plasmid pKB105 DNA (˜500 ng DNA, 2.5 μl) was digested in the following reaction: 4 μl 10×NEB Buffer 2, 1.5 μl BamHI (20 U/μl), 1.5 μl NheI (10 U/μl), and 30.5 μl autoclaved, deionized water, incubated at 37° C. for 3 hours. The digested DNA fragments were then isolated on 1.2% E-gels and purified using the QIAquick Gel Extraction Kits. DNA ligation reactions were prepared by combining 2 μl NheI/BamHI digested pKB105 (300 ng/nμl), 5 μl PCR fragment for ScoIIA or ScoIIB genes (100 ng/nμl), 2 μl 10× T4 DNA ligase buffer (New England Biolabs Cat. #MO202L), 1 μl T4 DNA ligase (400 U/μl), and 10 μl autoclaved, deionized water. The DNA ligation reactions were incubated overnight at 16° C.

The next day, 2 μL aliquots of the ligation reactions were used to transform E. coli TOP10 chemically competent cells (Invitrogen Corp.) following the manufacturer's protocol. Cells were plated on Luria Agar+50 μg/μl carbenicillin plates and incubated overnight at 37° C. The next day, 5 transformants from each set of plates were picked to inoculate culture tubes containing 5 ml Luria Broth+50 μg/μl carbenicillin Cultures were grown overnight at 37° C.

The next day, plasmid DNA was isolated using QIAquick Spin Miniprep Kits (Qiagen Inc.). Analytical DNA digestion using Nhe I and BamHI enzymes were performed to confirm that each clone had the expected insert size and vector backbone fragments.

Three independent clones showing the expected plasmid backbone and insert sizes were subjected to confirmatory DNA sequencing. When translated, the DNA sequences for the PCR amplified ScoIIA and ScoIIB coding regions of the genes were 100% identical to those previously reported (Gene ID: 1102951 for ScoIIA and Gene ID: 1097156 for ScoIIB).

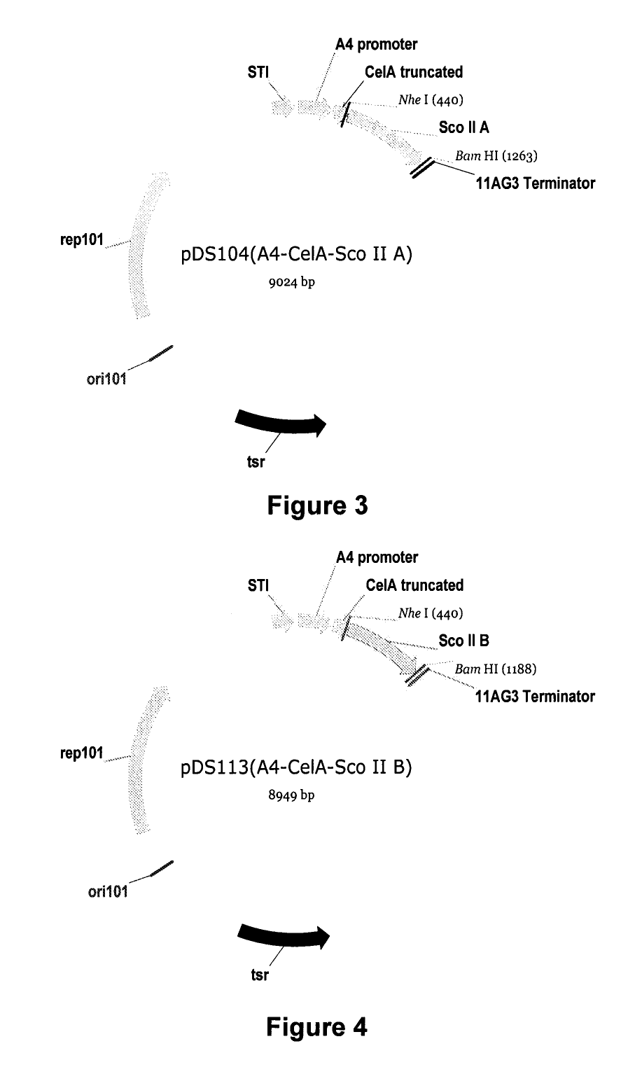

Following DNA sequence confirmation, the plasmids pDS104 (depicted in FIG. 3, for Sco II A) and pDS113 (depicted in FIG. 4, for Sco II B) were used to transform S. lividans TK23 derivative protoplasts, as described in U.S. Publication No. 2006/0154843.

The DNA sequence encoding the S. coelicolor ScoIIA gene (in plasmid pDS104) is shown as SEQ ID NO: 26. The ScoIIA coding sequence is shown in bold and the coding sequence for the CelA signal sequence is shown in normal typeface. The Nhe I restriction site is underlined. The DNA sequence encoding the S. coelicolor ScoIIB gene (in plasmid pDS113) is shown as SEQ ID NO: 27. The ScoIIB coding sequence is shown in bold and the coding sequence for the CelA signal sequence is shown in normal typeface. The Nhe I restriction site is underlined.

Transformation of Streptomyces lividans and Expression of ScoIIA and ScoIIB Proteins

Streptomyces lividans TK23 host cells were transformed with pDS104 and pDS113 plasmids. The protoplast transformation method described in Kieser et al., Practical Streptomyces Genetics, The John Innes Foundation, Norwich, United Kingdom (2000) was used. Transformed cells were plated on R5 selection plates and incubated at 30° C. for 3 days. One clone from the transformation plate was inoculated in TS G medium in shake flasks at 28° C. for 3 days. Cultures were then transferred to a Streptomyces 2 Modified Medium and incubated for an additional 4 days at 28° C. Supernatants were prepared by centrifugation of culture broths to obtain protein samples for further characterization.

### Example 4

### Cloning and Expression of Corynebacterium efficiens Enzyme with Lipase Activity (“CefII”)

Gene synthesis of Corynebacterium efficiens Cef II enzyme (NCBI: NP_{—}738716 BAC18916, ZP_{—}05750624; SEQ ID NO: 13) was performed based on a codon selection methods for improving expression in a Streptomyces lividans host. The CefII gene was synthesized and cloned into a vector named pGH [(XbaI)-A4 promoter (305 bp)-CelA truncated (138 bp, including a NheI site)-Cef II (837 bp)-Stop codon-BamHI-11AG3 terminator].

The Cef II gene containing DNA was digested with NheI and BamHI restriction endonucleases (New England Biolabs) as follows: 10 μl PCR product (100 ng/nμl), 2 μl 10×NEB Buffer 2 (New England Biolabs B7002S), 1 μl BamHI (20 U/μl), 1 μl NheI (10 U/μl), and 4 μl autoclaved, deionized water at 37° C. for 3 hours. Plasmid pKB105 DNA (−500 ng DNA, 2.5 μl) was digested in the following reaction: 2 μl 10×NEB Buffer 2, 1 μl BamHI (20 U/μl), 1 μl NheI (10 U/μl), and 13.5 μl autoclaved, deionized water at 37° C. for 3 hours. The digested DNA fragments were then isolated on 1.2% E-gels and purified using the QIAquick Gel Extraction Kit. DNA ligation reactions were prepared by combining 2 μl digested pKB105 (200 ng/μl), 5 μl Cef II fragment (200 ng/μl), 2 μl 10× T4 DNA ligase buffer (New England Biolab Cat #M0202L)), 1 μl T4 DNA ligase (400 U/μl), and 10 μl autoclaved, deionized water. The DNA ligation reactions were incubated overnight at 16° C.

The next day, 2 μL aliquots of the ligation reactions were used to transform E. coli TOP10 chemically competent cells (Invitrogen Corp.) following the manufacturer's protocol. Cells were plated on Luria Agar+50 μg/μl carbenicillin plates and incubated overnight at 37° C. The next day, 5 transformants from each set of plates were picked to inoculate cultures tubes of 5 ml Luria Broth+50 μg/μl carbenicillin. Cultures were grown overnight at 37° C.

The next day, plasmid DNA was isolated using QIAquick Spin Miniprep Kits (Qiagen Inc.). Analytical DNA digestion using Hind III and Xba I enzymes was performed to confirm that the clones had the expected insert size and vector backbone fragments. Three independent clones showing the expected plasmid backbone and insert size were subjected to confirmatory DNA sequencing. When translated, the DNA sequences for the PCR amplified CefII coding region of the gene was 100% identical to those previously reported (Gene ID: 1034874).

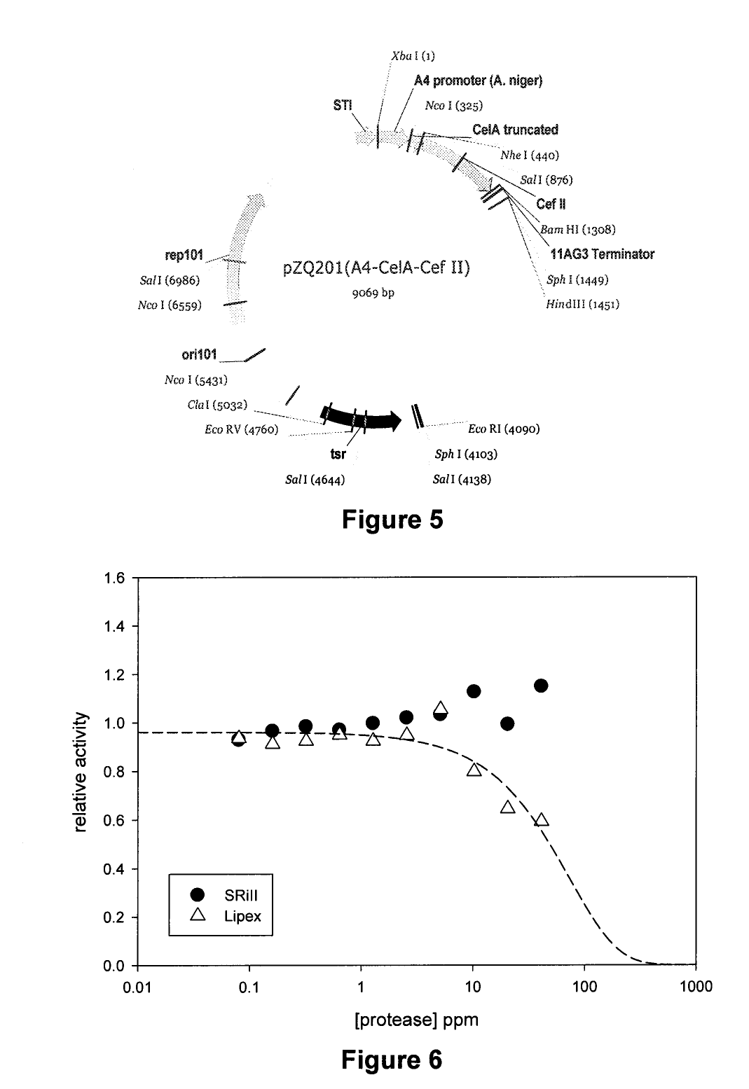

Following DNA sequencing confirmation, the plasmid pZQ201 (depicted in FIG. 5) was used to transform S. lividans TK23 derivative protoplasts, as described in U.S. Publication No. 2006/0154843. The DNA sequence for the Corynebacterium efficiens CefII gene sequence (as in plasmid pZQ201) is shown as SEQ ID NO: 28. The coding region of the Cef II gene is shown in bold. The coding sequence for the CelA signal sequence is shown in normal typeface. The Nhe I restriction site is underlined.

Transformation of Streptomyces lividans and Expression of Sco II A and Sco II B Proteins

The host Streptomyces lividans TK23 derivative strain was transformed with pZQ201 plasmid. The transformation technique was the protoplast method described in Kieser et al., “Practical Streptomyces Genetics,” The John Innes Foundation, Norwich, United Kingdom (2000). Transformed cells were plated on R5 selection plates and incubated at 30° C. for 3 days. One clone from the Streptomyces transformation plate was inoculated in TSG medium in shake flasks at 28° C. for 3 days. Cultures were then transferred to a Streptomyces 2 Modified Medium and incubated for an additional 4 days at 28° C. Supernatants were prepared by centrifugation of culture broths to obtain protein samples for further characterization.

### Example 5

### Stability of SriII Lipase Activity

### A. Stability in Detergent

Experiments to test the stability of SriII lipase activity in commercially available detergent were conducted. A 5% v/v solution of SriII lipase (purified, 20 mg/ml) in detergent (Laundry Dropps, Cot'n Wash Inc., Ardmore, Pa.) was incubated at room temperature. 10 μL of solution was removed at various time intervals over 1 week, serially diluted, and lipase activity measured with the pNB assay as described in Example 1. Remaining enzyme activity is reported as a fraction of activity measured at day 0 (Table 2).

TABLE 2
Stability of SriII lipase activity in Dropps detergent

| | Activity remaining | | |
|---|---|---|---|
| Days in Dropps detergent | 0 | 5 | 7 |
| SriII lipase | 1.00 | 0.85 | 0.55 |

### B. Stability in the Presence of Protease

Experiments to test the stability of SriII lipase activity in the presence of protease were conducted. A 200 ppm stock solution of SriII enzyme was prepared in 50 mM HEPES pH 8.2. 10 μL of serially diluted protease (B. amyloliquefaciens subtilisin BPN'-Y217L, Swissprot Accession Number P00782) in 50 mM HEPES pH 8.2 (protein concentration ranging from 0.1 to 100 ppm) was added to 100 μL of SriII enzyme in 96-well microtiter plates. The plates were incubated for 30 min at 30° C. Lipase activity remaining was measured with the pNB assay as described in Example 1. Relative activity of lipase was calculated by normalizing the rate of hydrolysis of pNB at the zero timepoint. SriII lipase activity exhibits enchanced stability when challenged by increasing amounts of protease, as compared to LIPEX™ (available from Novozymes) under the conditions tested (FIG. 6).

### Example 6

### Assessment of SriII, ScoIIB, and CefII Enzyme Activities

In this example, the ability of SriII, Sco II B or Cef II lipases to hydrolyse a variety of substrates (synthetic substrates, triglycerides, phospholipids, and lysophospholipids) was tested using assays described in Example 1.

A. pNB and pNPP Substrates

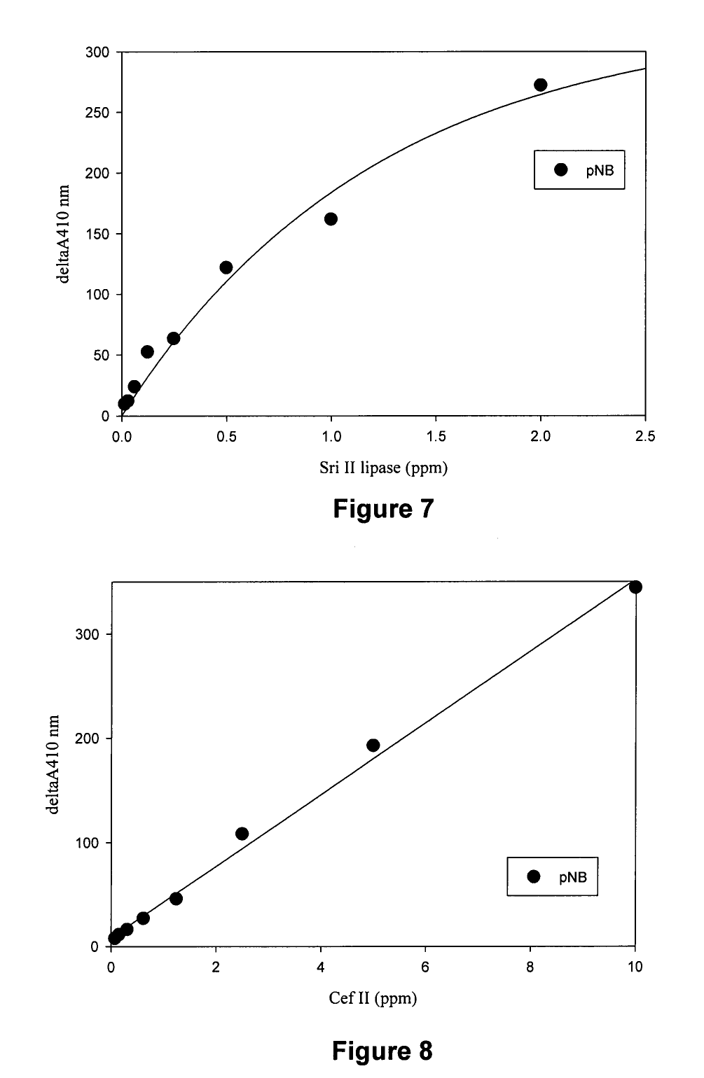

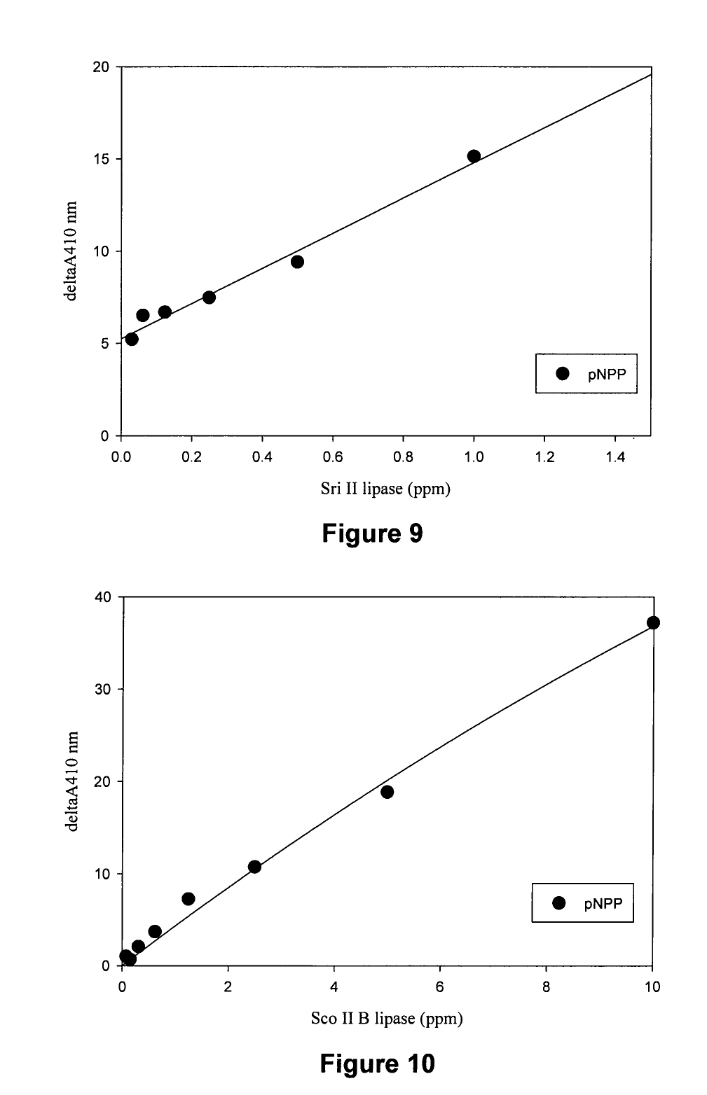

10 μL of serially diluted enzyme samples were incubated with 100 μL of substrate under conditions as indicated in the figures below. The release of products was kinetically measured using the pNB or pNPP assay as described in Example 1. The results for hydrolysis of pNB substrate are shown in FIGS. 7 and 8 and for hydrolysis of pNPP are shown in FIGS. 9 and 10.

### B. Triglycerides

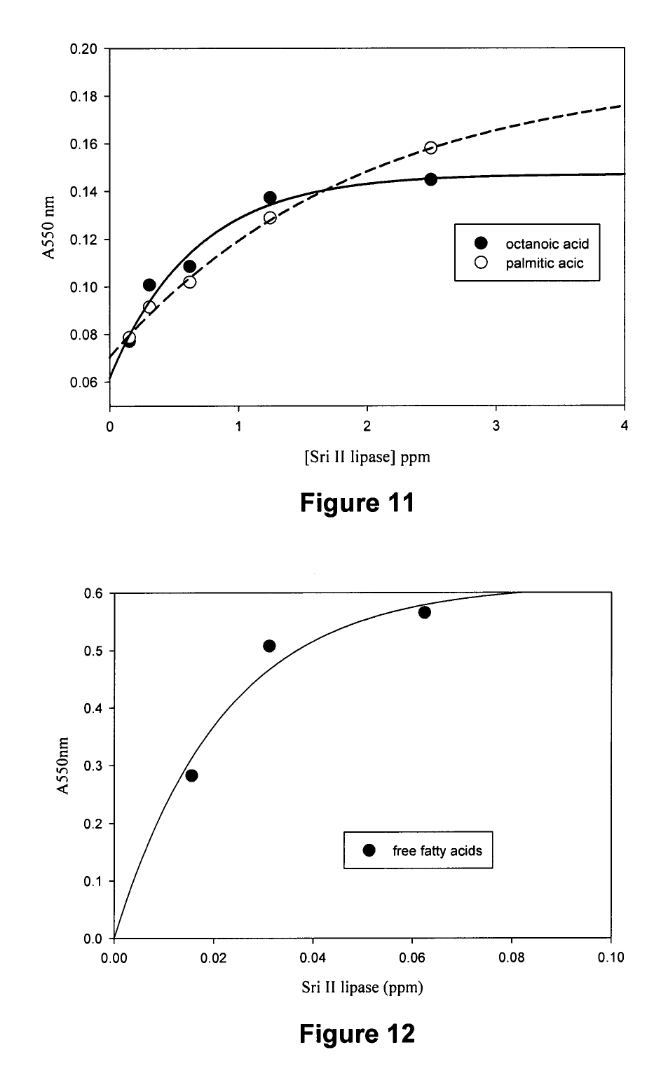

10 μL of serially diluted SriII lipase were incubated with glycerol trioctanoate and glyceryl tripalmitate in a 2% gum arabic emulsion. The release of products was measured using the Triglyceride hydrolysis assay to determine lipase activity in 96-well microtiter plates as described in Example 1. Hydrolysis of trioctanoate and tripalmitate by SriII lipase is shown in FIG. 11.

### C. Phospholipids

L-alpha-phosphotidylcholine at a concentration of 0.75% (w/v) was added to a buffer containing 2% polyvinyl alcohol, 50 mM HEPES, pH 8.2 and 6 gpg. The mixture was sonicated for 20 minutes with heat. The solution was clear. 20 ul of serially diluted enzyme samples were added to 100 ul of substrate. Reactions were incubated at 40° C. at 650 rpm for 3 hours. Phospholipid hydrolysis was assayed by measuring release of free fatty acids as described in “Assay to detect phospholipase activity in 96-well microtiter plates” in Example 1. Hydrolysis of L-alpha-phosphotidylcholine by SriII lipase and Sco II B lipase is shown in FIGS. 12 and 13, respectively.

### D. Lysophospholipids

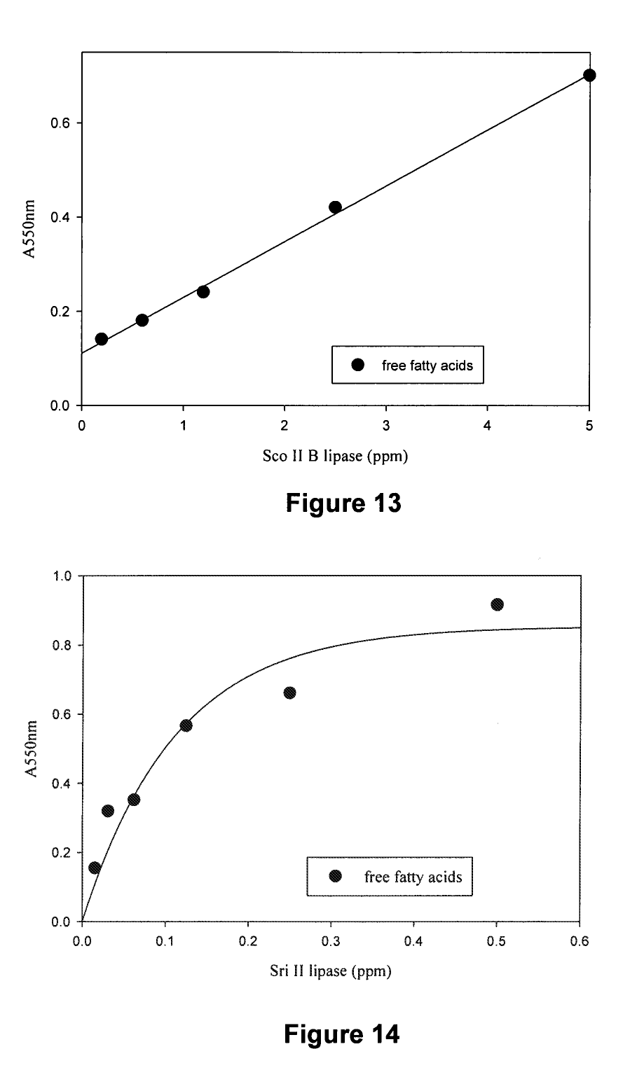

L-alpha-lysophosphatidylcholine at a concentration of 0.75% (w/v) was added to a buffer containing 2% polyvinyl alcohol (PVA), 50 mM HEPES, pH 8.2 and 6 gpg. The mixture was sonicated for 20 minutes with heat. The solution was clear. 20 ul of serially diluted SriII lipase were added to 100 ul of substrate. Reactions were incubated at 40° C., 650 rpm, for 16 hours. Lysophosphotidylcholine hydrolysis was assayed by measuring release of free fatty acids as described in “Assay to detect Lysophospholipase activity in 96-well microtiter plates” in Example 1. Hydrolysis of L-alpha-lysophosphatidylcholine by SriII is shown in FIG. 14.

### Example 7

### Hydrolysis of Triglycerides on Cloth by ScoIIA

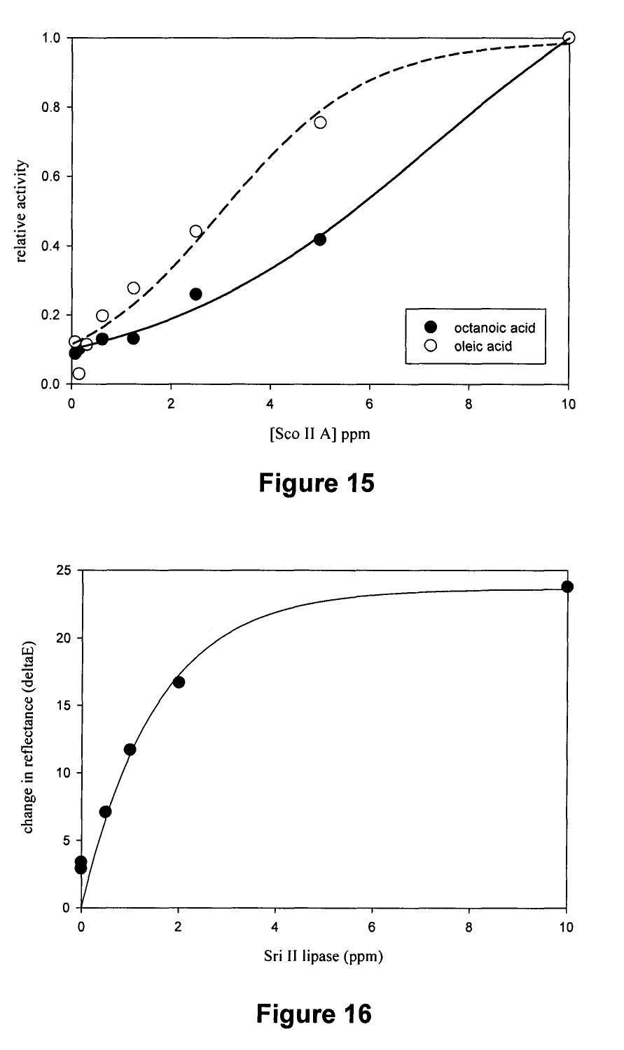

In this example, the ability of SriII to hydrolyse triglycerides bound to cloth was tested using the “Triglyceride hydrolysis assay on microswatches to determine lipase activity” assay as described in Example 1. Relative activity was calculated by normalizing the A550 nm signal in the NEFA assay to the highest concentration of enzyme for either trioctanoate or triolein. Hydrolysis of trioctanoic acid and triolein by SriII lipase is shown in FIG. 15.

### Example 8

### Enzyme Wash Performance of SriII

The enzyme wash performance of SriII was tested in Terg-o-tometer, and 12-well plate cleaning applications, using fabric swatches soiled with different lipid stains.

### A. Cleaning Performance Determination in Terg-O-Tometer Application

The enzyme wash performance of SriII lipase was measured in a Terg-o-tometer as described in Example 1. 20 ppm of purified SriII protein was added directly into 1 liter of the wash solution and reactions were then initiated by addition of 40 g/L of soiled (WFK 10 PF Pigment Vegetable Oil Stain) and ballast fabric. The washing reactions were agitated at 100 rpm for 2 hours at 40° C. Following cleaning, swatches were rinsed for 5 minutes in tap water, spun in a front-loading washing machine in the spin cycle for 7 minutes to remove excess water and air-dried. The control condition did not contain enzyme. Comparison of the extent of soil removal was assessed by reflectometry. For each enzyme tested, data is expressed as % soil removal index for enzyme vs. no enzyme (delta % SRI) (Table 3).

TABLE 3
Cleaning performance of SriII lipase on WFK 10 PF Pigment Vegetable
Oil Stain in terg-o-tometer application

| | Delta % SRI ± |
|---|---|
| | standard deviation |
| 20 ppm SriII | 9 ± 2% |
| 2 ppm LIPOMAX ™ | 10 ± 3% |
| (lipase from Pseudomonas alcaligenes) | |
| 1 ppm LIPEX ™ (Novozymes) | 2 ± 3% |

### B. Cleaning Performance Determination in 12-Well Plate Application

In this example, the stain removal performance of SriII lipase was measured using microswatches stained with oily soils in a 12-well plate format. 1.5 cm mini-swatches cut from oily swatches (Technical stains of lard on cotton dyed with Sudan Red, STC CFT CS-62 Lard with Sudan Red, Test Fabrics, Inc., West Pittiston, Pa.) were pre-read using a CROMA METER CR-200 Minolta reflectometer. One ml reactions were performed in the buffer (50 mM HEPES pH8.2, 6 gpg hardness) with concentrations of SriII ranging from 0.1 to 5 ppm. The reactions were incubated at 40° C. for 20 minutes. After incubation, the 1.5 cm mini-swatches were washed with distilled water and dried for 30 minutes at 60° C. Cleaning was calculated as the difference between the post- and pre-cleaning reflectometery measurements. A change in overall reflectance (deltaE) for each swatch is reported for n=6 (FIG. 16).

### Example 9

### Detection of Acyltransferase Activity of SriII

In this example, the acyltransferase activity of SriII lipase on a triglyceride substrate in solution or bound to cloth was measured by LC/MS analysis.

For assaying the acyltransferase activity, 20 μL of a 20 g/L triolein in 4% gum arabic emulsion was added to 50 mM phosphate buffer at pH 6 in 96-well microtiter plates. 8% (v/v) of acceptor (1,3-propanediol) was added to each well. Appropriately diluted enzyme solution (10 ppm) was added to the wells and the plate was incubated at 30° C. for 4 hours, with continuous mixing. After incubation, 100 μL of the supernatant was added to 900 μL of acetone in a microfuge tube and the contents spun in a microcentrifuge. After centrifugation, the supernatant was transferred to a fresh tube and was further diluted 3-fold with acetone, and 30 μL of this diluted supernatant was analyzed by LC/MS CAD (charged aerosol detection) analysis as described below.

Generic cotton microswatches, stained by the addition of 2 μL triolein were placed in 96-well microtiter plates. One hundred microliters of 100 mM phosphate buffer at pH 6, 10 μL of acceptor (1,3-propanediol) and 10 μL of serially diluted enzyme solution (10 ppm) was added to each well. The plates were incubated at 30° C. overnight with continuous mixing (platform shaker). The next day, the microswatches were removed from the plates, blotted dry and incubated with 200 μL of a 50:50 solution of acetone:hexane for extraction of soluble matter. The extract was diluted 20-fold with acetone and 30 μL of the diluted sample was analyzed by LC/MS CAD analysis as described below.

An Agilent 1100 (Hewlett Packard) HPLC was equipped with Alltima HP C18 column, 250×4.6 mm (Grace Davison). Compounds were eluted using a gradient beginning with solvent A (97% acetonitrile and 0.5% formic acid) with linearly increasing amounts of solvent B (neat acetone) over 10 minutes, followed by an isocratic phase in solvent B. The HPLC system was interfaced to an ABI 3200 QTrap MS (run under APCI mode), and a charged aerosol detector (ESA Biosciences) was used for quantification.

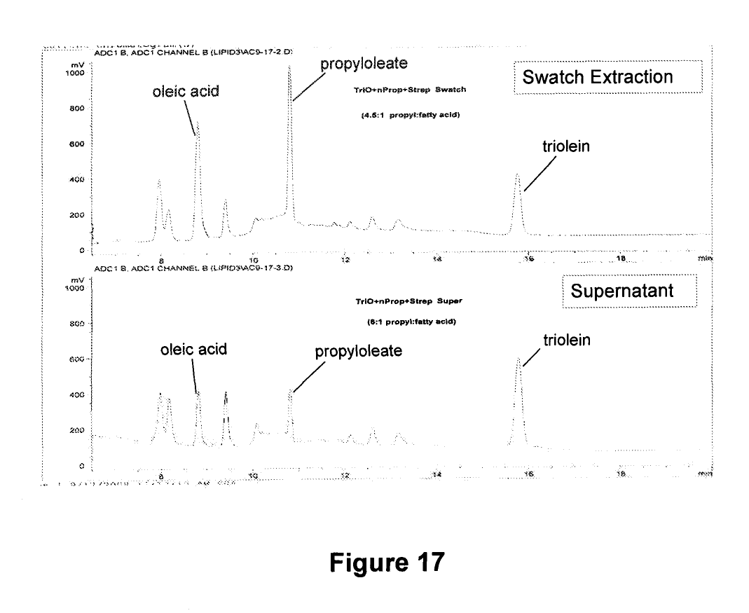

LC/MS CAD analysis (FIG. 17) of the mixtures obtained after overnight incubation of cloth-bound (top panel) or soluble substrate (lower panel) reactions with SriII using 1,3 propanediol as acceptor shows the formation of oleic acid and propyloleate.

### Example 10

### Peracetic Acid Generation

In this example, the ability of an enzyme as described herein to perform a perhydrolysis reaction (peracetic acid generation) is measured as described below.

Potassium phosphate buffer at pH 6.0 is prepared using standard methods. The reaction buffer consists of 2% (w/v, final concentration) poly(vinyl) alcohol (PVA; Sigma 341584) in 50 mM potassium phosphate solution buffered to pH 6.0. The substrate donors for the acyltransferase reaction are trioctanoate (Sigma T9126), triolein (Fluka 92860) or propylene glycol diacetate (Sigma 528072). These substrates are added to the 2% PVA solution at a final concentration of 0.75% (v/v). Emulsions are prepared by sonicating the donors in the PVA solutions for at least 20 minutes. Following formation of the emulsion, the acceptor, hydrogen peroxide (Sigma 516813), is added to the emulsions at a final concentration of 1% (v/v) hydrogen peroxide. Serial dilutions of enzyme are incubated with the reaction buffer which contains the emulsified donor and acceptors buffered to pH 6. Reactions are incubated for one hour at 25° C. Peracid generation is assayed by mixing the reaction products (20% v/v) in a peracid detection solution consisting of 1 mM 2,2′-azino-bis(3-ethylbenzthiazoline-6-sulfonic acid) (ABTS; Sigma A-1888), 500 mM glacial acetic acid pH 2.3, and 50 μM potassium iodide. The reaction of peracids with ABTS results in the generation of a radical cation ABTS⁺ which has an absorbance maximum around 400-420 nm. Peracid generation is assayed by measuring the absorbance at 420 nm of these reactions using a SpectraMax Plus³⁸⁴ microtiter plate reader.

### Listing of Amino Acid and Nucleotide Sequences

SEQ ID NO: 1: Amino acid sequence of SriII native full-length protein (Swissprot:
Q93MW7, Pubmed: AAK84028.1) (signal sequence shown in bold)
MRLSRRAATASALLLTPALALFGASAAVSAPRIQATDYVALGDSYSSGVGAGSY
DSSSGSCKRSTKSYPALWAASHTGTRFNFTACSGARTGDVLAKQLTPVNSGTDLVS
ITIGGNDAGFADTMTTCNLQGESACLARIAKARAYIQQTLPAQLDQVYDAIDSRAPA
AQVVVLGYPRFYKLGGSCAVGLSEKSRAAINAAADDINAVTAKRAADHGFAFGDV
NTTFAGHELCSGAPWLHSVTLPVENSYHPTANGQSKGYLPVLNSAT
SEQ ID NO: 2: Amino acid sequence of mature SriII protein
SAPRIQATDYVALGDSYSSGVGAGSYDSSSGSCKRSTKSYPALWAASHTGTRFNFT
ACSGARTGDVLAKQLTPVNSGTDLVSITIGGNDAGFADTMTTCNLQGESACLARIA
KARAYIQQTLPAQLDQVYDAIDSRAPAAQVVVLGYPRFYKLGGSCAVGLSEKSRA
AINAAADDINAVTAKRAADHGFAFGDVNTTFAGHELCSGAPWLHSVTLPVENSYH
PTANGQSKGYLPVLNSAT
SEQ ID NO: 3: Amino acid sequence of CelA signal sequence-SriII fusion protein
(CelA signal sequence is shown in bold)
MGFGSAPIALCPLRTRRNALKRLLALLATGVSIVGLTALAGPPAQASAPRIQAT
DYVALGDSYSSGVGAGSYDSSSGSCKRSTKSYPALWAASHTGTRFNFTACSGART
GDVLAKQLTPVNSGTDLVSITIGGNDAGFADTMTTCNLQGESACLARIAKARAYIQ
QTLPAQLDQVYDAIDSRAPAAQVVVLGYPRFYKLGGSCAVGLSEKSRAAINAAAD
DINAVTAKRAADHGFAFGDVNTTFAGHELCSGAPWLHSVTLPVENSYHPTANGQS
KGYLPVLNSAT
SEQ ID NO: 4: Nucleotide sequence for S. rimosus SriII gene (coding region is shown
as bold)
ATGCGCCTGTCCCGCCGCGCCGCCACCGCCTCCGCCCTGCTGCTGACCCCGGCC
CTGGCCCTGTTCGGCGCCTCCGCCGCCGTCAGCGCCCCGCGCATCCAGGCCAC
CGACTACGTGGCCCTCGGCGACTCCTACTCCTCGGGCGTGGGCGCCGGCT
CCTACGACAGCTCCAGCGGCTCCTGCAAGCGCTCCACCAAGTCCTACCCGG
CCCTGTGGGCCGCCTCCCACACCGGCACCCGCTTCAACTTCACCGCCTGCT
CCGGCGCCCGCACCGGCGACGTCCTGGCCAAGCAGCTGACCCCGGTCAAC
TCCGGCACCGACCTGGTCTCCATCACCATCGGCGGCAACGACGCCGGCTTC
GCCGACACCATGACCACCTGCAACCTGCAGGGCGAGTCCGCCTGCCTGGC
CCGCATCGCCAAGGCCCGCGCCTACATCCAGCAGACCCTGCCGGCCCAGC
TGGACCAGGTCTACGACGCCATCGACTCCCGCGCCCCGGCCGCGCAGGTC
GTCGTGCTGGGCTACCCGCGCTTCTACAAGCTGGGCGGCTCCTGCGCCGTC
GGCCTGTCCGAGAAGTCCCGCGCCGCCATCAACGCCGCCGCCGACGACAT
CAACGCCGTCACCGCCAAGCGCGCCGCCGACCACGGCTTCGCCTTCGGCG
ACGTCAACACCACCTTCGCCGGCCACGAGCTGTGCTCGGGCGCCCCGTGG
CTGCACTCCGTCACCCTCCCGGTCGAGAACAGCTACCACCCGACCGCCAAC
GGCCAGTCCAAGGGCTACCTGCCGGTCCTGAACTCCGCCACCTGA
SEQ ID NO: 5: Amino acid sequence of native full-length ScoIIA protein (NCBI:
NP_631558, CAC42140) (signal sequence shown in bold)
MPKPALRRVMTATVAAVGTLALGLTDATAHAAPAQATPTLDYVALGDSYSAGS
GVLPVDPANLLCLRSTANYPHVIADTTGARLTDVTCGAAQTADFTRAQYPGVAPQL
DALGTGTDLVTLTIGGNDNSTFINAITACGTAGVLSGGKGSPCKDRHGTSFDDEIEA
NTYPALKEALLGVRARAPHARVAALGYPWITPATADPSCFLKLPLAAGDVPYLRAI
QAHLNDAVRRAAEETGATYVDFSGVSDGHDACEAPGTRWIEPLLFGHSLVPVHPN
ALGERRMAEHTMDVLGLD
SEQ ID NO: 6: Amino acid sequence of mature ScoIIA protein
APAQATPTLDYVALGDSYSAGSGVLPVDPANLLCLRSTANYPHVIADTTGARLTDV
TCGAAQTADFTRAQYPGVAPQLDALGTGTDLVTLTIGGNDNSTFINAITACGTAGV
LSGGKGSPCKDRHGTSFDDEIEANTYPALKEALLGVRARAPHARVAALGYPWITPA
TADPSCFLKLPLAAGDVPYLRAIQAHLNDAVRRAAEETGATYVDFSGVSDGHDAC
EAPGTRWIEPLLFGHSLVPVHPNALGERRMAEHTMDVLGLD
SEQ ID NO: 7: Amino acid sequence of CelA signal sequence-ScoIIA fusion protein
(CelA signal sequence is shown in bold)
MGFGSAPIALCPLRTRRNALKRLLALLATGVSIVGLTALAGPPAQAAPAQATPT
LDYVALGDSYSAGSGVLPVDPANLLCLRSTANYPHVIADTTGARLTDVTCGAAQTA
DFTRAQYPGVAPQLDALGTGTDLVTLTIGGNDNSTFINAITACGTAGVLSGGKGSPC
KDRHGTSFDDEIEANTYPALKEALLGVRARAPHARVAALGYPWITPATADPSCFLK
LPLAAGDVPYLRAIQAHLNDAVRRAAEETGATYVDFSGVSDGHDACEAPGTRWIE
PLLFGHSLVPVHPNALGERRMAEHTMDVLGLD
SEQ ID NO: 8: Nucleotide sequence of ScoIIA gene (coding sequence is shown in bold)
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCGCGCCCGCCCAGGCCACTCCGA
CCCTGGACTACGTCGCCCTCGGCGACAGCTACAGCGCCGGCTCCGGCGTC
CTGCCCGTCGACCCCGCCAACCTGCTCTGTCTGCGCTCGACGGCCAACTAC
CCCCACGTCATCGCGGACACGACGGGCGCCCGCCTCACGGACGTCACCTG
CGGCGCCGCGCAGACCGCCGACTTCACGCGGGCCCAGTACCCGGGCGTCG
CACCCCAGTTGGACGCGCTCGGCACCGGCACGGACCTGGTCACGCTCACC
ATCGGCGGCAACGACAACAGCACCTTCATCAACGCCATCACGGCCTGCGGC
ACGGCGGGTGTCCTCAGCGGCGGCAAGGGCAGCCCCTGCAAGGACAGGCA
CGGCACCTCCTTCGACGACGAGATCGAGGCCAACACGTACCCCGCGCTCAA
GGAGGCGCTGCTCGGCGTCCGCGCCAGGGCTCCCCACGCCAGGGTGGCGG
CTCTCGGCTACCCGTGGATCACCCCGGCCACCGCCGACCCGTCCTGCTTCC
TGAAGCTCCCCCTCGCCGCCGGTGACGTGCCCTACCTGCGGGCCATCCAG
GCACACCTCAACGACGCGGTCCGGCGGGCCGCCGAGGAGACCGGAGCCAC
CTACGTGGACTTCTCCGGGGTGTCCGACGGCCACGACGCCTGCGAGGCCC
CCGGCACCCGCTGGATCGAACCGCTGCTCTTCGGGCACAGCCTCGTTCCCG
TCCACCCCAACGCCCTGGGCGAGCGGCGCATGGCCGAGCACACGATGGAC
GTCCTCGGCCTGGACTGA
SEQ ID NO: 9: Amino acid sequence of native full-length ScoIIB protein (NCBI:
NP_625998, CAB50940) (signal sequence shown in bold)
MRRFRLVGFLSSLVLAAGAALTGAATAQAAQPAAADGYVALGDSYSSGVGAGS
YISSSGDCKRSTKAHPYLWAAAHSPSTFDFTACSGARTGDVLSGQLGPLSSGTGLVS
ISIGGNDAGFADTMTTCVLQSESSCLSRIATAEAYVDSTLPGKLDGVYSAISDKAPN
AHVVVIGYPRFYKLGTTCIGLSETKRTAINKASDHLNTVLAQRAAAHGFTFGDVRT
TFTGHELCSGSPWLHSVNWLNIGESYHPTAAGQSGGYLPVLNGAA
SEQ ID NO: 10: Amino acid sequence of mature ScoIIB protein
AQPAAADGYVALGDSYSSGVGAGSYISSSGDCKRSTKAHPYLWAAAHSPSTFDFT
ACSGARTGDVLSGQLGPLSSGTGLVSISIGGNDAGFADTMTTCVLQSESSCLSRIATA
EAYVDSTLPGKLDGVYSAISDKAPNAHVVVIGYPRFYKLGTTCIGLSETKRTAINKA
SDHLNTVLAQRAAAHGFTFGDVRTTFTGHELCSGSPWLHSVNWLNIGESYHPTAA
GQSGGYLPVLNGAA
SEQ ID NO: 11: Amino acid sequence of CelA signal sequence-ScoIIB fusion protein
(CelA signal sequence is shown in bold)
MGFGSAPIALCPLRTRRNALKRLLALLATGVSIVGLTALAGPPAQAAQPAAAD
GYVALGDSYSSGVGAGSYISSSGDCKRSTKAHPYLWAAAHSPSTFDFTACSGARTG
DVLSGQLGPLSSGTGLVSISIGGNDAGFADTMTTCVLQSESSCLSRIATAEAYVDSTL
PGKLDGVYSAISDKAPNAHVVVIGYPRFYKLGTTCIGLSETKRTAINKASDHLNTVL
AQRAAAHGFTFGDVRTTFTGHELCSGSPWLHSVNWLNIGESYHPTAAGQSGGYLP
VLNGAA
SEQ ID NO: 12: Nucleotide sequence of ScoIIB gene (coding sequence is shown in
bold)
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCGCCCAACCCGCCGCCGCCGACG
GCTATGTGGCCCTCGGCGACTCCTACTCCTCCGGGGTCGGAGCGGGCAGC
TACATCAGCTCGAGCGGCGACTGCAAGCGCAGCACGAAGGCCCATCCCTA
CCTGTGGGCGGCCGCCCACTCGCCCTCCACGTTCGACTTCACCGCCTGTTC
CGGCGCCCGTACGGGTGATGTTCTCTCCGGACAGCTCGGCCCGCTCAGCTC
CGGCACCGGCCTCGTCTCGATCAGCATCGGCGGCAACGACGCCGGTTTCG
CCGACACCATGACGACCTGTGTGCTCCAGTCCGAGAGCTCCTGCCTGTCGC
GGATCGCCACCGCCGAGGCGTACGTCGACTCGACGCTGCCCGGCAAGCTC
GACGGCGTCTACTCGGCAATCAGCGACAAGGCGCCGAACGCCCACGTCGT
CGTCATCGGCTACCCGCGCTTCTACAAGCTCGGCACCACCTGCATCGGCCT
GTCCGAGACCAAGCGGACGGCGATCAACAAGGCCTCCGACCACCTCAACA
CCGTCCTCGCCCAGCGCGCCGCCGCCCACGGCTTCACCTTCGGCGACGTAC
GCACCACCTTCACCGGCCACGAGCTGTGCTCCGGCAGCCCCTGGCTGCACA
GCGTCAACTGGCTGAACATCGGCGAGTCGTACCACCCCACCGCGGCCGGC
CAGTCCGGTGGCTACCTGCCGGTCCTCAACGGCGCCGCCTGA
SEQ ID NO: 13: Amino acid sequence of native full-length CefII protein (NCBI:
NP_738716, BAC18916) (signal sequence is shown in bold)
MRTTVIAASALLLLAGCADGAREETAGAPPGESSGGIREEGAEASTSITDVYIALG
DSYAAMGGRDQPLRGEPFCLRSSGNYPELLHAEVTDLTCQGAVTGDLLEPRTLGER
TLPAQVDALTEDTTLVTLSIGGNDLGFGEVAGCIRERIAGENADDCVDLLGETIGEQ
LDQLPPQLDRVHEAIRDRAGDAQVVVTGYLPLVSAGDCPELGDVSEADRRWAVEL
TGQINETVREAAERHDALFVLPDDADEHTSCAPPQQRWADIQGQQTDAYPLHPTSA
GHEAMAAAVRDALGLEPVQP
SEQ ID NO: 14: Amino acid sequence of mature CefII protein
REETAGAPPGESSGGIREEGAEASTSITDVYIALGDSYAAMGGRDQPLRGEPFCLRSS
GNYPELLHAEVTDLTCQGAVTGDLLEPRTLGERTLPAQVDALTEDTTLVTLSIGGND
LGFGEVAGCIRERIAGENADDCVDLLGETIGEQLDQLPPQLDRVHEAIRDRAGDAQV
VVTGYLPLVSAGDCPELGDVSEADRRWAVELTGQINETVREAAERHDALFVLPDD
ADEHTSCAPPQQRWADIQGQQTDAYPLHPTSAGHEAMAAAVRDALGLEPVQP
SEQ ID NO: 15: Amino acid sequence of CelA signal sequence-Cef II fusion protein
(CelA signal sequence is shown in bold)
MGFGSAPIALCPLRTRRNALKRLLALLATGVSIVGLTALAGPPAQAREETAGAP
PGESSGGIREEGAEASTSITDVYIALGDSYAAMGGRDQPLRGEPFCLRSSGNYPELLH
AEVTDLTCQGAVTGDLLEPRTLGERTLPAQVDALTEDTTLVTLSIGGNDLGFGEVA
GCIRERIAGENADDCVDLLGETIGEQLDQLPPQLDRVHEAIRDRAGDAQVVVTGYLP
LVSAGDCPELGDVSEADRRWAVELTGQINETVREAAERHDALFVLPDDADEHTSC
APPQQRWADIQGQQTDAYPLHPTSAGHEAMAAAVRDALGLEPVQP
SEQ ID NO: 16: Nucleotide sequence of Cef II gene (coding sequence is shown in bold)
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCCGCGAGGAAACCGCCGGCGCGC
CACCGGGCGAGTCGTCGGGGGAATCCGAGAAGAGGGAGCTGAGGCCTCCA
CCAGCATCACCGACGTCTACATCGCCCTCGGCGATTCGTACGCCGCGATGG
GTGGGCGCGACCAGCCCCTGCGCGGGGAGCCCTTCTGCCTCCGCAGTTCG
GGTAACTACCCCGAGCTGCTTCACGCGGAGGTGACGGACCTCACGTGCCA
GGGCGCGGTCACCGGCGACCTGTTGGAGCCGCGGACTCTGGGCGAGCGCA
CCCTGCCGGCGCAGGTGGACGCGCTGACGGAGGACACCACGCTGGTCACC
CTCAGCATCGGCGGGAACGACCTCGGCTTCGGGGAGGTCGCCGGCTGTAT
CCGCGAGCGCATCGCCGGCGAGAACGCAGATGACTGCGTCGACCTGCTCG
GCGAGACCATCGGCGAACAGCTGGACCAGCTCCCGCCCCAGCTGGACCGG
GTGCACGAGGCCATCCGGGACCGCGCCGGCGACGCGCAAGTCGTGGTCAC
CGGTTACCTGCCGCTGGTGTCAGCCGGCGACTGCCCGGAACTCGGCGACG
TCTCCGAGGCCGACAGGCGTTGGGCCGTCGAACTCACCGGCCAGATCAAC
GAGACAGTACGGGAGGCGGCCGAGCGCCATGACGCCCTGTTCGTGCTGCC
CGACGACGCCGACGAGCACACCAGCTGCGCCCCCCCGCAGCAGCGGTGGG
CAGACATTCAGGGCCAGCAGACGGACGCCTACCCCCTGCACCCGACGTCC
GCGGGCCACGAAGCAATGGCTGCGGCCGTCCGGGACGCGCTGGGACTCGA
GCCGGTGCAGCCTTGA
SEQ ID NO: 17: Primer EL-917 (+)
GCGATCCTCTAGAGATCGAACTTCATGTTCGAG
SEQ ID NO: 18: Primer EL-920 (−)
GCTTATAAGCTTCATCAGGTGGCGGAGTTCAGGAC
SEQ ID NO: 19: Primer EL-921 (−)
GGTGGCCTGGATGCGCGGGGCGCTGGCCTGTGCCGGGGGGCCGGCTAG
SEQ ID NO: 20: Primer EL-922 (+)
CTAGCCGGCCCCCCGGCACAGGCCAGCGCCCCGCGCATCCAGGCCACC
SEQ ID NO: 21: Primer Sdf5
AGCGCTAGCCGGCCCCCCGGCACAGGCCGCGCCCGCCCAGGCCACTCCGACC
SEQ ID NO: 22: Primer Sdf6
TCCGGATCCAGG TCAGTCCAGGCCGAGGACGTCCATC
SEQ ID NO: 23: Primer 1725-Fw
AGCGCTAGCCGGCCCCCCGGCACAGGCCGCCCAACCCGCCGCCGCCGACGGC
SEQ ID NO: 24: Primer 1725-Rv
TCCGGATCCAGGTCA GGCGGCGCCGTTGAGG
SEQ ID NO: 25: The DNA sequence for the coding region of the SriII gene in plasmid
Strep lipase B.
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCAGCGCCCCGCGCATCCAGGCCA
CCGACTACGTGGCCCTCGGCGACTCCTACTCCTCGGGCGTGGGCGCCGGC
TCCTACGACAGCTCCAGCGGCTCCTGCAAGCGCTCCACCAAGTCCTACCCG
GCCCTGTGGGCCGCCTCCCACACCGGCACCCGCTTCAACTTCACCGCCTGC
TCCGGCGCCCGCACCGGCGACGTCCTGGCCAAGCAGCTGACCCCGGTCAA
CTCCGGCACCGACCTGGTCTCCATCACCATCGGCGGCAACGACGCCGGCTT
CGCCGACACCATGACCACCTGCAACCTGCAGGGCGAGTCCGCCTGCCTGG
CCCGCATCGCCAAGGCCCGCGCCTACATCCAGCAGACCCTGCCGGCCCAG
CTGGACCAGGTCTACGACGCCATCGACTCCCGCGCCCCGGCCGCGCAGGT
CGTCGTGCTGGGCTACCCGCGCTTCTACAAGCTGGGCGGCTCCTGCGCCGT
CGGCCTGTCCGAGAAGTCCCGCGCCGCCATCAACGCCGCCGCCGACGACA
TCAACGCCGTCACCGCCAAGCGCGCCGCCGACCACGGCTTCGCCTTCGGC
GACGTCAACACCACCTTCGCCGGCCACGAGCTGTGCTCGGGCGCCCCGTG
GCTGCACTCCGTCACCCTCCCGGTCGAGAACAGCTACCACCCGACCGCCAA
CGGCCAGTCCAAGGGCTACCTGCCGGTCCTGAACTCCGCCACCTGA
SEQ ID NO: 26: The DNA sequence encoding the S. coelicolor ScoIIA gene (in plasmid
pDS104)
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCGCGCCCGCCCAGGCCACTCCGA
CCCTGGACTACGTCGCCCTCGGCGACAGCTACAGCGCCGGCTCCGGCGTC
CTGCCCGTCGACCCCGCCAACCTGCTCTGTCTGCGCTCGACGGCCAACTAC
CCCCACGTCATCGCGGACACGACGGGCGCCCGCCTCACGGACGTCACCTG
CGGCGCCGCGCAGACCGCCGACTTCACGCGGGCCCAGTACCCGGGCGTCG
CACCCCAGTTGGACGCGCTCGGCACCGGCACGGACCTGGTCACGCTCACC
ATCGGCGGCAACGACAACAGCACCTTCATCAACGCCATCACGGCCTGCGGC
ACGGCGGGTGTCCTCAGCGGCGGCAAGGGCAGCCCCTGCAAGGACAGGCA
CGGCACCTCCTTCGACGACGAGATCGAGGCCAACACGTACCCCGCGCTCAA
GGAGGCGCTGCTCGGCGTCCGCGCCAGGGCTCCCCACGCCAGGGTGGCGG
CTCTCGGCTACCCGTGGATCACCCCGGCCACCGCCGACCCGTCCTGCTTCC
TGAAGCTCCCCCTCGCCGCCGGTGACGTGCCCTACCTGCGGGCCATCCAG
GCACACCTCAACGACGCGGTCCGGCGGGCCGCCGAGGAGACCGGAGCCAC
CTACGTGGACTTCTCCGGGGTGTCCGACGGCCACGACGCCTGCGAGGCCC
CCGGCACCCGCTGGATCGAACCGCTGCTCTTCGGGCACAGCCTCGTTCCCG
TCCACCCCAACGCCCTGGGCGAGCGGCGCATGGCCGAGCACACGATGGAC
GTCCTCGGCCTGGACTGA
SEQ ID NO: 27: The DNA sequence encoding the S. coelicolor ScoIIB gene (in plasmid
pDS 113)
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCGCCCAACCCGCCGCCGCCGACG
GCTATGTGGCCCTCGGCGACTCCTACTCCTCCGGGGTCGGAGCGGGCAGC
TACATCAGCTCGAGCGGCGACTGCAAGCGCAGCACGAAGGCCCATCCCTA
CCTGTGGGCGGCCGCCCACTCGCCCTCCACGTTCGACTTCACCGCCTGTTC
CGGCGCCCGTACGGGTGATGTTCTCTCCGGACAGCTCGGCCCGCTCAGCTC
CGGCACCGGCCTCGTCTCGATCAGCATCGGCGGCAACGACGCCGGTTTCG
CCGACACCATGACGACCTGTGTGCTCCAGTCCGAGAGCTCCTGCCTGTCGC
GGATCGCCACCGCCGAGGCGTACGTCGACTCGACGCTGCCCGGCAAGCTC
GACGGCGTCTACTCGGCAATCAGCGACAAGGCGCCGAACGCCCACGTCGT
CGTCATCGGCTACCCGCGCTTCTACAAGCTCGGCACCACCTGCATCGGCCT
GTCCGAGACCAAGCGGACGGCGATCAACAAGGCCTCCGACCACCTCAACA
CCGTCCTCGCCCAGCGCGCCGCCGCCCACGGCTTCACCTTCGGCGACGTAC
GCACCACCTTCACCGGCCACGAGCTGTGCTCCGGCAGCCCCTGGCTGCACA
GCGTCAACTGGCTGAACATCGGCGAGTCGTACCACCCCACCGCGGCCGGC
CAGTCCGGTGGCTACCTGCCGGTCCTCAACGGCGCCGCCTGA
SEQ ID NO: 28: The DNA sequence for the Corynebacterium efficiens CefII gene
sequence (as in plasmid pZQ201)
ATGGGCTTTGGGAGCGCTCCCATCGCGTTGTGTCCGCTTCGCACGAGGAGGAAC
GCTTTGAAACGCCTTTTGGCCCTGCTCGCGACCGGCGTGTCGATCGTCGGCCTG
ACTGCGCTAGCCGGCCCCCCGGCACAGGCCCGCGAGGAAACCGCCGGCGCGC
CACCGGGCGAGTCGTCGGGGGAATCCGAGAAGAGGGAGCTGAGGCCTCCA
CCAGCATCACCGACGTCTACATCGCCCTCGGCGATTCGTACGCCGCGATGG
GTGGGCGCGACCAGCCCCTGCGCGGGGAGCCCTTCTGCCTCCGCAGTTCG
GGTAACTACCCCGAGCTGCTTCACGCGGAGGTGACGGACCTCACGTGCCA
GGGCGCGGTCACCGGCGACCTGTTGGAGCCGCGGACTCTGGGCGAGCGCA
CCCTGCCGGCGCAGGTGGACGCGCTGACGGAGGACACCACGCTGGTCACC
CTCAGCATCGGCGGGAACGACCTCGGCTTCGGGGAGGTCGCCGGCTGTAT
CCGCGAGCGCATCGCCGGCGAGAACGCAGATGACTGCGTCGACCTGCTCG
GCGAGACCATCGGCGAACAGCTGGACCAGCTCCCGCCCCAGCTGGACCGG
GTGCACGAGGCCATCCGGGACCGCGCCGGCGACGCGCAAGTCGTGGTCAC
CGGTTACCTGCCGCTGGTGTCAGCCGGCGACTGCCCGGAACTCGGCGACG
TCTCCGAGGCCGACAGGCGTTGGGCCGTCGAACTCACCGGCCAGATCAAC
GAGACAGTACGGGAGGCGGCCGAGCGCCATGACGCCCTGTTCGTGCTGCC
CGACGACGCCGACGAGCACACCAGCTGCGCCCCCCCGCAGCAGCGGTGGG
CAGACATTCAGGGCCAGCAGACGGACGCCTACCCCCTGCACCCGACGTCC
GCGGGCCACGAAGCAATGGCTGCGGCCGTCCGGGACGCGCTGGGACTCGA
GCCGGTGCAGCCTTGA

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A detergent composition comprising a polypeptide selected from the group consisting of SriII, ScoIIA, ScoIIB, CefII, and a variant, thereof, wherein the detergent composition exhibits improved cleaning of an oily stain compared to an equivalent detergent composition lacking the polypeptide.

2. The detergent composition of claim 1, wherein the polypeptide is SriII.

3. The detergent composition of claim 1, wherein the polypeptide is ScoIIA.

4. The detergent composition of claim 1, wherein the polypeptide is ScoIIB.

5. The detergent composition of claim 1, wherein the polypeptide is CefII.

6. The detergent composition of claim 1, wherein the polypeptide is SriII and the polypeptide comprises an amino acid sequence that is at least 90% identical to the amino acid sequence of SEQ ID NO: 2.

7. The detergent composition of claim 1, wherein the polypeptide is ScoIIA and the polypeptide comprises an amino acid sequence that is at least 90% identical to the amino acid sequence of SEQ ID NO: 6.

8. The detergent composition of claim 1, wherein the polypeptide is ScoIIB and the polypeptide comprises an amino acid sequence that is at least 90% identical to the amino acid sequence of SEQ ID NO: 10.

9. The detergent composition of claim 1, wherein the polypeptide is CefII and the polypeptide comprises an amino acid sequence that is at least 90% identical to the amino acid sequence of SEQ ID NO: 14.

10. A detergent composition comprising a polypeptide having at least 90% amino acid sequence identity to a polypeptide comprising an amino acid sequence selected from the group consisting of SEQ ID NO: 2, SEQ ID NO: 6, SEQ ID NO: 10, SEQ ID NO: 14, wherein the detergent composition exhibits improved cleaning of an oily stain compared to an equivalent detergent composition lacking the polypeptide.

11. The detergent composition of claim 10, wherein the polypeptide has at least 90% amino acid sequence identity to a polypeptide comprising an amino acid sequence of SEQ ID NO: 2.

12. The detergent composition of claim 10, wherein the polypeptide has at least 90% amino acid sequence identity to a polypeptide comprising an amino acid sequence of SEQ ID NO: 6.

13. The detergent composition of claim 10, wherein the polypeptide has at least 90% amino acid sequence identity to a polypeptide comprising an amino acid sequence of SEQ ID NO: 10.

14. The detergent composition of claim 10, wherein the polypeptide has at least 90% amino acid sequence identity to a polypeptide comprising an amino acid sequence of SEQ ID NO: 14.

15. The detergent composition of claim 1, wherein the polypeptide has lipase enzymatic activity and at least one additional activity selected from phospholipase, lysophospholipase, and acyltransferase activity.

16. The detergent composition of claim 1, wherein the composition comprises at least one surfactant.

17. The detergent composition of claim 1, wherein the composition comprises at least one additional polypeptide selected from the group consisting of a protease, an amylase, a cellulase, a laccase, a lipase, a phospholipase, a lysophospholipase, an acyltransferase, a perhydrolase, and an arylesterase.

18. A method for cleaning an oily stain on a fabric, comprising contacting the stain with a detergent composition of claim 1 under wash conditions in which the polypeptide is enzymatically active, wherein catalytic action of the polypeptide on a component of the stain facilitates removal of at least a portion of the stain from the fabric.

19. The method of claim 18, wherein the oily stain comprises triglycerides.
